(19) United States
(12) Patent Application Publication
(10) Pub. No.: US 2023/0095395 A1
(43) Pub. Date: Mar. 30, 2023

(54) MULTIPLEX IMMUNOFLUORESCENCE DETECTION OF TARGET ANTIGENS

(71) Applicant: AUCKLAND UNISERVICES LIMITED, Auckland (NZ)

(72) Inventors: Peter Roderick Dunbar, Auckland (NZ); Saemmul Park, Auckland (NZ)

(21) Appl. No.: 17/801,386

(22) PCT Filed: Feb. 25, 2021
(86) PCT No.: PCT/IB2021/051581

§ 371 Date: Aug. 22, 2022

(30) Foreign Application Priority Data
Feb. 28, 2020 (AU) 2020900589

(51) Int. Cl. G01N 33/574

(52) U.S. Cl. CPC G01N 33/57484

(57) ABSTRACT

A method of multispectral immunofluorescence imaging of a biological sample is described. The described method allows direct detection of seven or more target antigens simultaneously using directly labeled antibody fluorophore conjugates. The described method enables multiplex detection and analysis of a plurality of biomarkers simultaneously across an entire planar biological sample, providing unique spatio-temporal insights in immune-therapeutics and immuno-diagnostics.

### FIELD OF THE INVENTION

This invention generally relates to simultaneous visualization of a plurality of target antigens in a biological sample including methods and reagents related thereto.

### BACKGROUND

Multispectral/high dimensional imaging has taken on increasing importance for practitioners in biomedical research and in clinical medicine/pathology. The ability to visualize multiple, specific molecules within a tissue sample provides a powerful tool for both research and clinical medicine applications. For example, this ability allows the spatial arrangement of different cell types to be determined, having applications for both health and disease management and treatment.

In medicine, detection of target molecules, particularly proteins, that serve as biomolecular markers or “biomarkers” within tissue samples is desirable in helping to identify the type of therapy that a patient is likely to respond to. In one example patients with a cancer arising in a particular tissue may be grouped for different therapy according to the biomarkers that are detected within that tissue. In pathology it may also be necessary to quantify the number of cells expressing a particular target protein before a recommendation for a particular therapy can be made.

Immunohistochemical techniques currently in use will typically detect a single target protein in any one tissue section of a tissue sample. For many diseases (such as breast cancer) this means several tissue sections must be labeled with different antibodies before a recommendation for optimal therapy can be made.

Immunofluorescence microscopy (IFM) techniques currently employed can detect more than one molecule simultaneously in a single tissue section from a sample. However, current techniques typically employ indirect labeling using unconjugated primary antibodies that are subsequently labeled using different secondary antibodies conjugated to different fluorophores (FPs), each FP having different emission spectra. This secondary labeling process allows the target proteins labeled by various antibodies to be detected and localized individually within the same tissue section.

Unfortunately, while IFM is used in both biomedical research and in some clinical pathology contexts, IFM has usually been limited to the detection of two to three, maximally four different colours; i.e., the detection of two to three different antibody labels and a nuclear stain. This limitation has been due to traditional fluorescent microscopes having only the ability to separate certain emission spectra of different fluorophores.

The use of multispectral imaging has recently improved the number of fluorophores that can be distinguished in a single tissue section. For example, the Opal staining platform can label up to nine different target antigens in a single section (Gorris, 2018). However, there are major drawbacks related to the use of current IFM labeling protocols in multispectral, multiplex imaging of tissue sections, including the intensity of labour and length of time required to complete the protocols.

Current multiplex labeling techniques are labour intensive. For example, the Opal staining technique typically takes 3-5 days to complete a multi-colour stain. These platforms are also very difficult to iterate, meaning it will often take months to develop a new multi-colour staining protocol. In the case of Opal, considerable knowledge of IFM and the particular markers of interest are required for the development of new and/or modified panels.

Accordingly, there is a need in both research and clinical medicine for new and improved methods of multiplex immunofluorescence detection of a plurality of target molecules from a single tissue sample.

It is an object of the invention to go at least some way towards addressing the above deficiencies in the prior art by providing a method of multiplex immunofluorescence detection of a plurality of target molecules from a single tissue sample including reagents used there, and/or to at least provide the public with a useful choice.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### SUMMARY OF THE INVENTION

In one aspect the present invention relates to a composition comprising at least three, four, five, six or at least seven antibody-fluorophore conjugates (Ab-FP), wherein each FP has a different maximum fluorescence excitation and emission wavelength (Ex).

In another aspect the invention relates to a method of direct immunofluorescence analysis of biological sample comprising
a) labeling at least one target antigen in a planar sample of the biological sample with at least one unique Ab-FP conjugate, and
b) generating a multispectral fluorescence image of the labeled planar sample using a multispectral scanner, wherein the image comprises at least two colours, wherein at least one colour is associated with the specific binding of the at least one unique Ab-FP conjugate to the at least one target antigen, and
c) determining from the image the presence or absence of a biomarker comprising the at least one target antigen.

In another aspect the invention relates to a multispectral immunofluorescence image of a planar biological sample, the image comprising at least three, preferably four, five, six, seven, preferably at least eight colours, wherein at least three, preferably four, five, six, preferably seven colours are associated with the specific binding of at least three, preferably four, five, six, preferably seven Ab-FPs to target antigens comprised in the planar sample.

In another aspect the invention relates to a method of detecting a plurality of target antigens in a biological sample comprising
a) simultaneously labeling at least two target antigens in a planar sample of the biological sample with at least two unique Ab-FP conjugates, wherein the at least two target antigens are present on or in a cell in the sample, and
b) generating a multispectral fluorescence image of the labeled planar sample using a multispectral scanner, wherein the image comprises at least three colours, wherein at least two colours are associated with the specific binding of each unique Ab-FP conjugate to a target antigen, and
c) determining from the image, the presence or absence of the at least two target antigens, each target antigen labeled with a different unique Ab-FP.

In another aspect the invention relates to a method of detecting a plurality of biomarkers in a biological sample comprising
a) simultaneously labeling at least two target antigens in a planar sample of the biological sample with at least two unique Ab-FP conjugates, wherein each target antigen is comprised by a biomarker in the sample, and
b) generating a multispectral fluorescence image of the labeled planar sample using a multispectral scanner, wherein the image comprises at least three colours, wherein at least two colours are associated with the specific binding of each unique Ab-FP conjugate to a target antigen, and
c) determining from the image, the presence or absence of a plurality of biomarkers, each biomarker comprising a target antigen labeled with a different unique Ab-FP.

In another aspect the invention relates to a method of detecting a plurality of different cell types in a biological sample comprising
a) simultaneously labeling at least two target antigens in a planar sample with at least two unique Ab-FP conjugates, wherein the target antigens are present on or in a cell in the sample, and
b) generating a multispectral fluorescence image of the labeled planar sample using a multispectral scanner, wherein the image comprises at least three colours, wherein at least two colours are associated with the specific binding of each unique Ab-FP conjugate to a target antigen on a different cell type, and
c) determining from the image, the presence or absence of at least two cell types, each cell type labeled with a different unique Ab-FP.

In another aspect the invention relates to a method of identifying the abundance of a plurality of cell types in a biological sample comprising:
a) simultaneously labeling a planar biological sample with at least two, preferably three, four, five, six, preferably at least seven unique antibody-fluorophore conjugates (Ab-FP), wherein each Ab-FP specifically binds a target antigen on or in a different cell, and
b) generating a multispectral image of the labeled planar sample by simultaneously detecting the fluorescence emission spectra of each FP from each Ab-FP respectively, and
c) determining the abundance of a plurality of different cell types in the planar sample based on the fluorescence emission spectra detected, optionally with reference to a suitable reference control.

In another aspect the invention relates to a method determining the spatial distribution of a plurality of cell types in a biological sample comprising
a) simultaneously labeling at least two target antigens in a planar sample of the biological sample with at least two unique Ab-FP conjugates, wherein each of the at least two target antigens is present on or in a different cell-type in the sample,
b) generating a multispectral fluorescence image of the labeled planar sample using a multispectral scanner, wherein the image comprises at least three colours, wherein at least two colours are associated with the specific binding of each unique Ab-FP conjugate to a target antigen,
c) identifying from the image at least two different cell types based on the binding of each Ab-FP to the at least two target antigens, and
d) determining from the image, the spatial distribution of a plurality of cell types in a biological sample.

In another aspect the invention relates to a method of identifying a patient sub-group from within a group of patients comprising:
a) simultaneously labeling at least three, preferably four, five, six, preferably seven different biomarkers in a planar biological sample from a patient with at least two, preferably three, four, five, six, preferably seven unique antibody-fluorophore conjugates (Ab-FP), wherein each Ab-FP specifically binds a target antigen on a biomarker,
b) generating a multispectral image of the labeled planar sample section by simultaneously detecting the fluorescence emission spectra of each FP in each Ab-FP respectively,
c) detecting the presence or abundance each biomarker in the image generated in b), wherein each biomarker is identified in the image as a different colour that is associated with the specific binding of a unique Ab-FP, and
d) determining from the image that a patient is in a sub-group based on the presence or abundance of each Ab-FP that is specifically bound to each biomarker.

In another aspect the invention relates to a method of making a diagnostic panel of antibody-fluorophore conjugates (Ab-FPs) comprising:
a) identifying at least three, preferably four, five, six, preferably seven biomarkers for a pre-determined disease or condition,
b) obtaining a unique Ab-FP for each biomarker identified, each Ab-FP comprising an antibody that specifically binds a target antigen on one of the biomarkers identified in a), preferably on each biomarker identified in a), each Ab-FP having a fluorophore (FP) having a maximum fluorescence emission wavelength of about 420 nm to about 850 nm,
c) simultaneously labeling a planar biological sample with the Ab-FPs in b), wherein labeling comprises specifically binding each Ab-FP to a biomarker, preferably wherein each Ab-FP binds a different biomarker, respectively,
d) obtaining a multispectral image of the fluorescence emission spectra of each FP,
e) identifying in the multispectral image the presence or abundance of each biomarker, wherein each biomarker is identified in the image as a different colour that is associated with the specific binding of a different Ab-FP to a target antigen on a biomarker, and
f) selecting the unique Ab-FP conjugates that can be identified in the image in e) as a panel of Ab-FPs that are diagnostic for the pre-determined disease or condition in a).

In another aspect, the invention relates to a method of identifying a replacement antibody-fluorophore (Ab-FP) conjugate for direct immunofluorescence analysis of a biological sample comprising:
a) generating a first multispectral immunofluorescence image from a single planar biological sample using a first set of at least two to at least seven unique Ab-FP conjugates following a method as described herein, the first multispectral immunofluorescence image comprising up to eight different colours, wherein up to seven colours are each associated with a unique Ab-FP conjugate,
b) selecting at least one of the Ab-FP conjugates from the first set for replacement with a replacement Ab-FP,
c) identifying a suitable antibody for the replacement Ab-FP
d) identifying a suitable fluorophore for the replacement Ab-FP
e) obtaining a replacement Ab-FP,
f) replacing the Ab-FP selected in b) with the replacement Ab-FP to generate a second set of at least two to at least seven unique Ab-FP conjugates,
g) generating a second immunofluorescence image using the second set of Ab-FP conjugates according to a method as described herein, and
h) comparing the first and second multispectral immunofluorescence images,

wherein no difference in the ability to discriminate each different colour in the multispectral image when the first and second images are compared in h) confirms the identification of the replacement Ab-FP conjugate.

In another aspect, the invention relates to a method for determining if at least one cell type is responsive to a candidate drug; the method comprising:
a) determining the abundance or spatial distribution of at least one cell type in a planar biological sample comprising the at least one cell type using a multispectral immunofluorescence detection method as described herein, and
b) determining if the at least one cell type is responsive to the candidate drug based on the abundance or spatial distribution of the at least one cell type in the sample, optionally in comparison to a suitable control.

In one embodiment the planar biological sample comprises cells cultured in vitro. In one embodiment the planar biological sample comprises a biological tissue or portion thereof.

In another aspect the invention relates to a method for predicting a treatment response of a patient to a proposed treatment for a pre-determined disease or condition comprising
a) determining the abundance or spatial distribution of at least one cell type in a planar biological sample from the patient using a multispectral immunofluorescence detection method as herein, and
b) determining that the patient will or will not be responsive to the proposed treatment based on the abundance or spatial distribution of the at least one cell type in the sample, optionally in comparison to a suitable control.

In another aspect the invention relates to a method for identifying a cellular response to a candidate drug comprising
a) contacting a planar biological sample containing a plurality of cells with the candidate drug,
b) determining the abundance or spatial distribution of at least one cell type in the sample using a multispectral immunofluorescence detection method as herein, and
c) determining from the abundance or spatial distribution of the at least one cell type in sample that there is a cellular response to the candidate drug, optionally by comparison to a suitable control.

In another aspect the invention relates to a method of identifying a patient that will benefit from a candidate therapy comprising:
a) labeling a planar biological sample obtained from the subject with at least one unique Ab-FP conjugate, preferably with from one to 12 unique Ab-FP conjugates,
b) obtaining at least one digital fluorescence image of the labeled sample using a multispectral scanner;
c) extracting data associated with at least one emission spectra associated with an Ab-FP conjugate,
d) calculating a distribution function which captures the distribution of data for the at least one emission spectra;
e) deriving a summary score for a patient from the distribution function;
f) evaluating the summary score relative to at least one reference value;

selecting the subject as a candidate for a specified therapy based on the summary score, and
g) optionally treating the subject with the specified therapy.

In another aspect the invention relates to a method of detecting a plurality of biomarkers in a biological sample comprising
a) simultaneously labeling at least seven target antigens in a planar sample of the biological sample with at least two unique Ab-FP conjugates, wherein each target antigen is comprised by a biomarker in the sample, and
b) generating a multispectral fluorescence image of the labeled planar sample using a multispectral scanner, wherein the image comprises at least eight colours, wherein at least seven colours are associated with the specific binding of each unique Ab-FP conjugate to a target antigen, and
c) determining from the image, the presence or absence of a plurality of biomarkers, each biomarker comprising a target antigen labeled with a different unique Ab-FP.

Various embodiments of the different aspects of the invention as discussed above are also set out below in the detailed description of the invention, but the invention is not limited thereto.

Other aspects of the invention may become apparent from the following description which is given by way of example only and with reference to the accompanying drawings.

### BRIEF DESCRIPTION OF THE DRAWINGS

The invention will now be described with reference to the figures in the accompanying drawings.

FIG. 1—Unmixed image (greyscale) of Ab-FP+DAPI labeled melanoma-infiltrated lymph node tissue showing CD3 expression

Unmixed image of one of seven colours (6 Ab-FP+DAPI) detected simultaneously from an Ab-FP labeled melanoma-infiltrated lymph node tissue section showing the distribution of CD3+ T cells only. CD3+ T cells labeled with an anti-CD3-AF532 antibody conjugate are shown as bright and/or grey.

FIG. 2—Unmixed image (greyscale) of Ab-FP+DAPI labeled melanoma-infiltrated lymph node tissue showing CD21 expression

Unmixed image of one of seven colours (6 Ab-FP+DAPI) detected simultaneously from an Ab-FP labeled melanoma-infiltrated lymph node tissue section showing the distribution of CD21+B cells and follicular dendritic cells only. CD21+B cells and follicular dendritic cells labeled with an anti-CD21-BB700 antibody conjugate are shown as bright and/or grey.

FIG. 3—Unmixed image (greyscale) of Ab-FP+DAPI labeled melanoma-infiltrated lymph node tissue showing CD31 expression

Unmixed image of one of seven colours (6 Ab-FP+DAPI) detected simultaneously from an Ab-FP labeled melanoma-infiltrated lymph node tissue showing the distribution of CD31+ blood and lymphatic endothelial cells only. CD31+ blood and lymphatic endothelial cells labeled with an anti-CD31-BV480 antibody conjugate are shown as bright and/or grey.

FIG. 4—Unmixed image (greyscale) of Ab-FP+DAPI labeled melanoma-infiltrated lymph node tissue showing CD34 expression

Unmixed image of one of seven colours (6 Ab-FP+DAPI) detected simultaneously from an Ab-FP labeled melanoma-infiltrated lymph node tissue section showing the distribution of CD34+ blood and lymphatic endothelial cells only. CD34+ blood and lymphatic endothelial cells labeled with an anti-CD34-PE-CF594 antibody conjugate are shown as bright and/or grey.

FIG. 5—Unmixed image (greyscale) of Ab-FP+DAPI labeled melanoma-infiltrated lymph node tissue showing CD141 expression

Unmixed image of one of seven colours (6 Ab-FP+DAPI) detected simultaneously from an Ab-FP labeled melanoma-infiltrated lymph node tissue section showing the distribution of CD141+ marginal reticular cells and dendritic cells only. CD141+ marginal reticular cells and dendritic cells labeled with an anti-CD141-BB515 antibody conjugate are shown as bright and/or grey.

FIG. 6—Unmixed image (greyscale) of Ab-FP+DAPI labeled melanoma-infiltrated lymph node tissue showing Ki67 expression

Unmixed image of one of seven colours (6 Ab-FP+DAPI) detected simultaneously from an Ab-FP labeled melanoma-infiltrated lymph node tissue section showing the distribution of Ki67+ cells only. Ki67+ cells labeled with an anti-Ki67-AF647 antibody conjugate are shown as bright and/or grey.

FIG. 7—Unmixed image (greyscale) of Ab-FP+DAPI labeled melanoma-infiltrated lymph node tissue showing the nuclear stain DAPI only

Unmixed image of one of seven colours (6 Ab-FP+DAPI) detected simultaneously from an Ab-FP labeled melanoma-infiltrated lymph node tissue section showing the nuclear stain DAPI only.

FIG. 8—Combined image (greyscale) of Ab-FP+DAPI labeled melanoma-infiltrated lymph node tissue.

The combination of the unmixed images in FIGS. 1 to 7 showing all seven colours (6 Ab-FP+DAPI) detected simultaneously from an Ab-FP labeled melanoma-infiltrated lymph node tissue section.

FIG. 9—Combined image (greyscale) of Ab-FP labeled melanoma-infiltrated lymph node tissue without DAPI.

The combination of the unmixed images in FIGS. 1 to 6 showing six of seven colours (6 Ab-FP+DAPI) detected simultaneously from an Ab-FP labeled melanoma-infiltrated lymph node tissue section. DAPI is not shown.

FIG. 10—Combined image (colour) of Ab-FP+DAPI labeled melanoma-infiltrated lymph node tissue.

The combination of the unmixed images in FIGS. 1 to 7 showing all seven colours (6 Ab-FP+DAPI) detected simultaneously from an Ab-FP labeled melanoma-infiltrated lymph node tissue section.

FIG. 11—Combined image (colour) of Ab-FP labeled melanoma-infiltrated lymph node tissue without DAPI.

The combination of the unmixed images in FIGS. 1 to 6 showing six of seven colours (6 Ab-FP+DAPI) detected simultaneously from an Ab-FP labeled melanoma-infiltrated lymph node tissue section. DAPI is not shown.

FIG. 12—Combined image (greyscale) of Ab-FP labeled melanoma-infiltrated lymph node tissue+DAPI.

Combined unmixed image of seven colours (6 Ab-FP+DAPI) detected simultaneously from an Ab-FP labeled melanoma-infiltrated lymph node tissue section. The image shows the distribution of T cells, B cells, FDCs (follicular dendritic cells), MRCs (marginal reticular cells), BECs (blood endothelial cells), LECs (lymphatic endothelial cells), and Ki67+ cells as shown in FIGS. 1 to 6 respectively. DAPI indicates nuclear stain.

FIG. 13—Combined image (greyscale) of Ab-FP labeled melanoma-infiltrated lymph node tissue without DAPI.

Combined unmixed image of six of seven colours (6 Ab-FP+DAPI) detected simultaneously from an Ab-FP labeled melanoma-infiltrated lymph node tissue section. The image shows the distribution of T cells, B cells, FDCs (follicular dendritic cells), MRCs (marginal reticular cells), BECs (blood endothelial cells), LECs (lymphatic endothelial cells), and Ki67+ cells as shown in FIGS. 1 to 6 respectively. DAPI is not shown.

FIG. 14—Unmixed image (greyscale) of Ab-FP+DAPI labeled melanoma-infiltrated lymph node tissue showing DAPI only.

Unmixed image of one of eight colours (7 Ab-FP+DAPI) detected simultaneously from an Ab-FP labeled melanoma-infiltrated lymph node tissue section showing the nuclear stain DAPI only.

FIG. 15—Unmixed image (greyscale) of Ab-FP+DAPI labeled melanoma-infiltrated lymph node tissue showing CD31 expression.

Unmixed image of one of eight colours (7 Ab-FP+DAPI) detected simultaneously from an Ab-FP labeled melanoma-infiltrated lymph node tissue section showing the distribution of CD31+ blood and lymphatic endothelial cells only. CD31+ blood and lymphatic endothelial cells labeled with an anti-CD31-BV480 antibody conjugate are shown as bright and/or grey.

FIG. 16—Unmixed image (greyscale) of Ab-FP+DAPI labeled melanoma-infiltrated lymph node tissue showing CD141 expression.

Unmixed image of one of eight colours (7 Ab-FP+DAPI) detected simultaneously from an Ab-FP labeled melanoma-infiltrated lymph node tissue section showing the distribution of CD141+ marginal reticular cells and dendritic cells only. CD141+ marginal reticular cells and dendritic cells labeled with an anti-CD141-BB515 antibody conjugate are shown as bright and/or grey.

FIG. 17—Unmixed image (greyscale) of Ab-FP+DAPI labeled melanoma-infiltrated lymph node tissue showing CD3 expression.

Unmixed image of one of eight colours (7 Ab-FP+DAPI) detected simultaneously from an Ab-FP labeled melanoma-infiltrated lymph node tissue section showing the distribution of CD3+ T cells only. CD3+ T cells labeled with an anti-CD3-AF532 antibody conjugate are shown as bright and/or grey.

FIG. 18—Unmixed image (greyscale) of Ab-FP+DAPI labeled melanoma-infiltrated lymph node tissue showing CD34 expression.

Unmixed image of one of eight colours (7 Ab-FP+DAPI) detected simultaneously from an Ab-FP labeled melanoma-infiltrated lymph node tissue section showing the distribution of CD34+ blood and lymphatic endothelial cells only. CD34+ blood and lymphatic endothelial cells labeled with an anti-CD34-PE-CF594 antibody conjugate are shown as bright and/or grey

FIG. 19—Unmixed image (greyscale) of Ab-FP+DAPI labeled melanoma-infiltrated lymph node tissue showing Ki67 expression.

Unmixed image of one of eight colours (7 Ab-FP+DAPI) detected simultaneously from an Ab-FP labeled melanoma-infiltrated lymph node tissue section showing the distribution of Ki67+ cells only. Ki67+ cells labeled with an anti-Ki67-AF647 antibody conjugate are shown as bright and/or grey.

FIG. 20—Unmixed image (greyscale) of Ab-FP+DAPI labeled melanoma-infiltrated lymph node tissue showing CD21 expression.

Unmixed image of one of eight colours (7 Ab-FP+DAPI) detected simultaneously from an Ab-FP labeled melanoma-infiltrated lymph node tissue section showing the distribution of CD21+B cells and follicular dendritic cells only. CD21+B cells and follicular dendritic cells labeled with an anti-CD21-BB700 antibody conjugate are shown as bright and/or grey.

FIG. 21—Unmixed image (greyscale) of Ab-FP+DAPI labeled melanoma-infiltrated lymph node tissue showing CD11c expression.

Unmixed image of one of eight colours (7 Ab-FP+DAPI) detected simultaneously from an Ab-FP labeled melanoma-infiltrated lymph node tissue section showing the distribution of CD11c+ cells only. CD11c+ cells labeled with an anti-CD11c-AF700 antibody conjugate are shown as bright and/or grey.

FIG. 22—Combined image (greyscale) of Ab-FP+DAPI labeled melanoma-infiltrated lymph node tissue showing 8-colours combined.

The combination of the unmixed images in FIGS. 14 to 21 showing all eight colours (7 Ab-FP+DAPI) detected simultaneously from an Ab-FP labeled melanoma-infiltrated lymph node tissue section. Combined unmixed image shows the distribution of T cells, B cells, FDCs (follicular dendritic cells), MRCs (marginal reticular cells), BECs (blood endothelial cells), LECs (lymphatic endothelial cells), CD11c+ cells, and Ki67+ cells. DAPI indicates nuclear stain.

FIG. 23—Combined image (greyscale) of Ab-FP+DAPI labeled melanoma-infiltrated lymph node tissue showing 7 colours combined (DAPI not shown).

The combination of the unmixed images in FIGS. 14 to 21 showing seven colours (7 Ab-FP+DAPI where DAPI is not shown) detected simultaneously from an Ab-FP labeled melanoma-infiltrated lymph node tissue section. Combined unmixed image shows the distribution of T cells, B cells, FDCs (follicular dendritic cells), MRCs (marginal reticular cells), BECs (blood endothelial cells), LECs (lymphatic endothelial cells), CD11c+ cells, and Ki67+ cells.

FIG. 24—Unmixed image of melanoma-infiltrated lymph node tissue section showing CD163 expression.

Unmixed image of melanoma-infiltrated lymph node tissue stained with anti-CD163 APC/Fire750 showing the distribution of CD163+ cells. CD163+ cells labeled with an anti-CD163-APC/Fire750 antibody conjugate are shown as bright and/or grey.

FIG. 25—Unmixed image of melanoma-infiltrated lymph node tissue section showing CD163 expression.

Unmixed image of melanoma-infiltrated lymph node tissue stained with anti-CD163 APC/Fire750 showing the distribution of CD163+ cells. CD163+ cells labeled with an anti-CD163-APC/Fire750 antibody conjugate are shown as bright and/or grey.

FIG. 26—Unmixed image (greyscale) showing CD19 expression (with or without DAPI): AF647

Unmixed images of 2-colour (CD19-AF647+DAPI) stained FFPE tonsil tissue showing the distribution of CD19+ B cells (detected by anti-CD19 AF647 antibody), with (A) or without (B) DAPI.

CD19+ B cells labeled with an anti-CD19-AF647 antibody conjugate are shown as bright and/or grey.

FIG. 27—Unmixed image showing CD8 expression (with or without DAPI): AF647

Unmixed images of 2-colour (CD8-AF647+DAPI) stained FFPE tonsil tissue showing the distribution of CD8+ T cells (detected by anti-CD8 AF647 antibody), with (A) or without (B) DAPI.

CD8+ T cells labeled with an anti-CD8 AF647 antibody conjugate are shown as bright and/or grey.

FIG. 28—Unmixed image showing CD45RO expression (with or without DAPI): AF488

Unmixed images of 2-colour (CD45RO-AF488+DAPI) stained FFPE tonsil tissue showing the distribution of CD45RO+ activated and memory T cells and some B cells (detected by anti-CD45RO AF488 antibody), with (A) or without (B) DAPI.

CD45RO+ activated, memory T cells and some B cells labeled with an anti-CD45RO AF488 antibody conjugate are shown as bright and/or grey.

FIG. 29—Unmixed image showing CD45RO expression (with or without DAPI): AF594

Unmixed images of 2-colour (CD45-AF594+DAPI) stained FFPE tonsil tissue showing the distribution of CD45RO+ activated and memory T cells and some B cells (detected by anti-CD45RO AF594 antibody), with (A) or without (B) DAPI.

CD45RO+ activated, memory T cells and some B cells labeled with an anti-CD45RO AF488 antibody conjugate are shown as bright and/or grey.

FIG. 30—Unmixed image showing CD45RO expression (with or without DAPI): AF700

Unmixed images of 2-colours (CD45-AF700+DAPI) stained FFPE tonsil tissue showing the distribution of CD45RO+ activated and memory T cells and some B cells (detected by anti-CD45RO AF700 antibody), with (A) or without (B) DAPI.

CD45RO+ activated, memory T cells and some B cells labeled with an anti-CD45RO AF488 antibody conjugate are shown as bright and/or grey.

FIG. 31—Unmixed image showing CD45RO expression (with or without DAPI): BV510

Unmixed images of 2-colours (CD45-BV510+DAPI) stained FFPE tonsil tissue showing the distribution of CD45RO+ activated and memory T cells and some B cells (detected by anti-CD45RO BV510 antibody), with (A) or without (B) DAPI.

CD45RO+ activated, memory T cells and some B cells labeled with an anti-CD45RO AF488 antibody conjugate are shown as bright and/or grey.

FIG. 32—Unmixed image showing CD45RO expression (with or without DAPI):BV650

Unmixed images of 2-colours (CD45-BV650+DAPI) stained FFPE tonsil tissue showing the distribution of CD45RO+ activated and memory T cells and some B cells (detected by anti-CD45RO BV650 antibody), with (A) or without (B) DAPI.

FIG. 33—Unmixed image showing CD45RO expression (with or without DAPI): PE/Dazzle 594

Unmixed images of 2-colours (CD45-PE/Dazzle 594+DAPI) stained FFPE tonsil tissue showing the distribution of CD45RO+ activated and memory T cells and some B cells (detected by anti-CD45RO PE/Dazzle 594 antibody), with (A) or without (B) DAPI.

CD45RO+ activated, memory T cells and some B cells labeled with an anti-CD45RO AF488 antibody conjugate are shown as bright and/or grey.

FIG. 34—Unmixed image showing CD45RO expression (with or without DAPI): APC/Fire750

Unmixed images of 2-colours (CD45-APC/Fire750+DAPI) stained FFPE tonsil tissue showing the distribution of CD45RO+ activated and memory T cells and some B cells (detected by anti-CD45RO APC/Fire750 antibody), with (A) or without (B) DAPI.

FIG. 35—Unmixed image showing expression of CD19 and CD45RO

Combined unmixed image of 3-colours (2 Abs+DAPI) stained FFPE tonsil tissue (A) showing the distribution of CD19+ B cells (detected by anti-CD19-AF647 antibody(B)), and CD45RO+ activated and memory T cells and some B cells (detected by anti-CD45RO AF488 antibody(C)) and DAPI (D).

FIG. 36—Unmixed image showing expression of CD8 and CD45RO

Combined unmixed image of 3-colours (2 Abs+DAPI) stained FFPE tonsil tissue (A) showing the distribution of CD8+ T cells (detected by anti-CD8-AF647 antibody (B)), and CD45RO+ activated and memory T cells and some B cells (detected by anti-CD45RO AF488 antibody (C)) and DAPI (D).

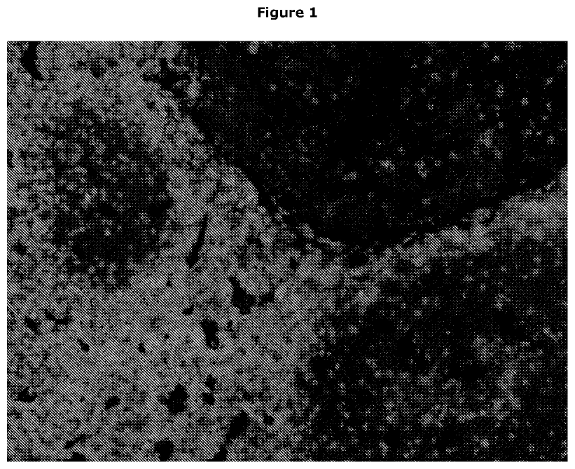

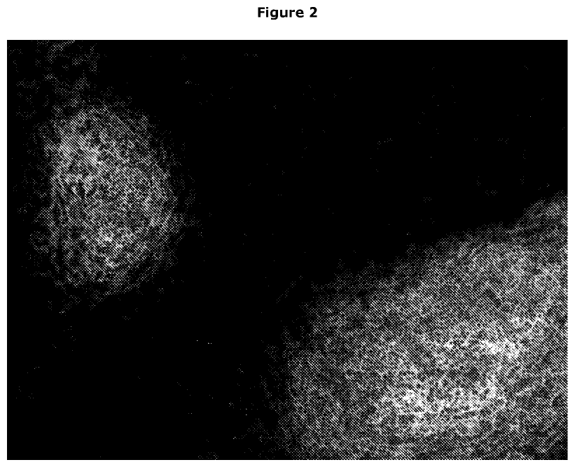

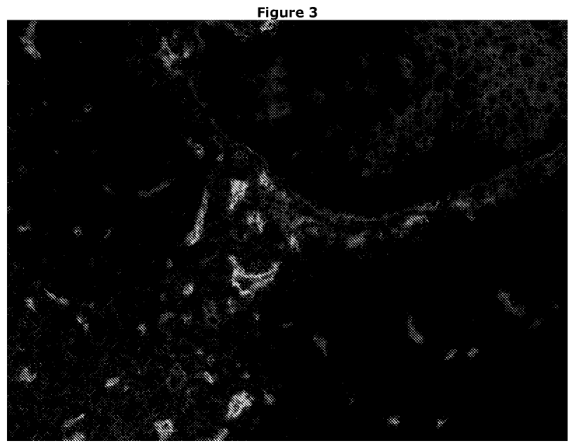

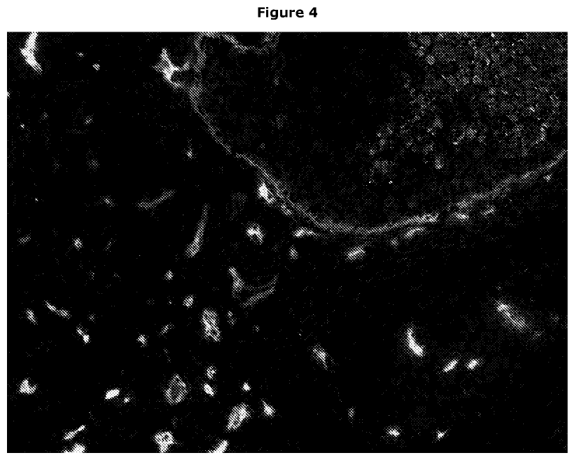

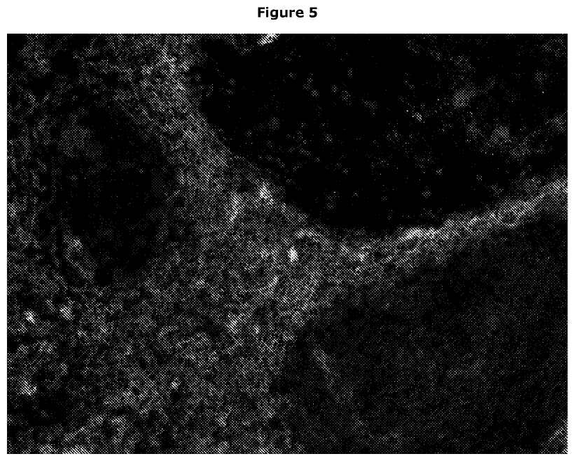

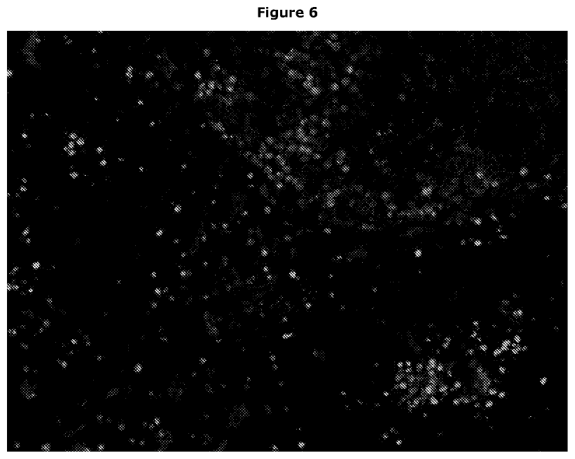

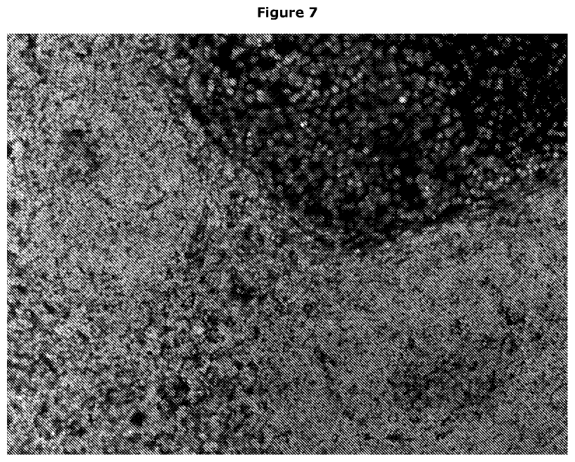

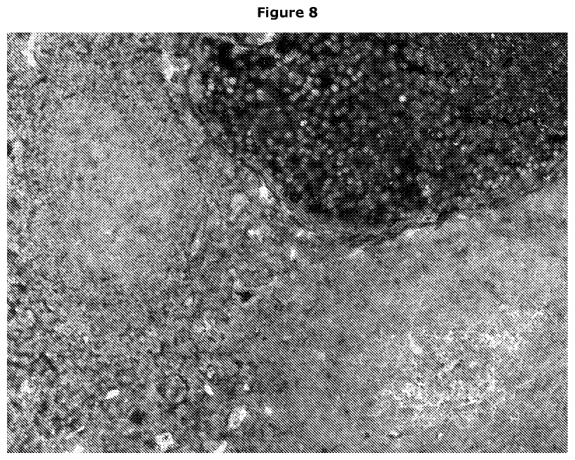

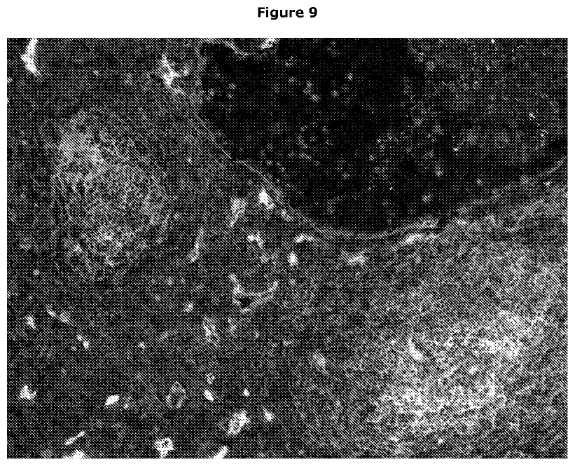

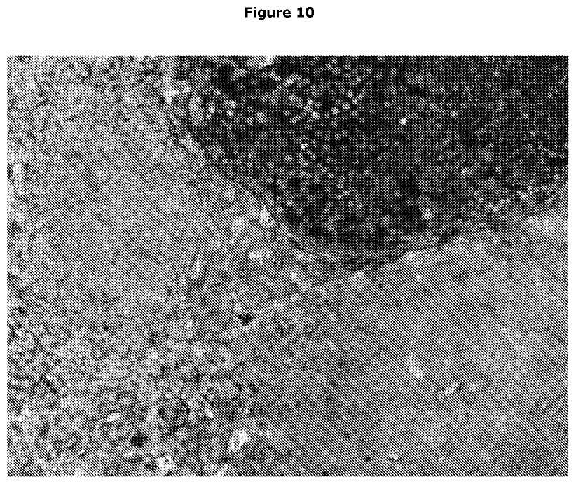

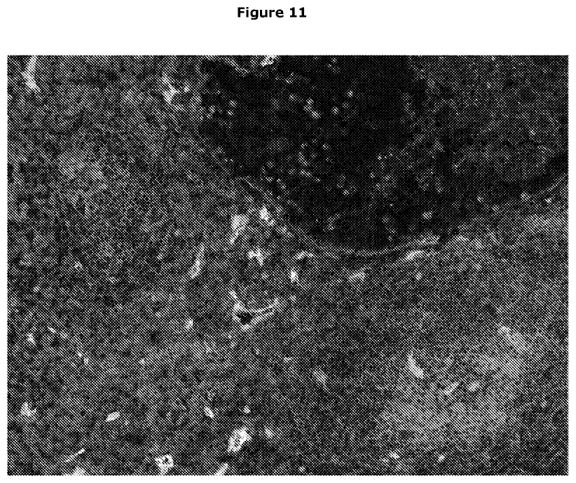

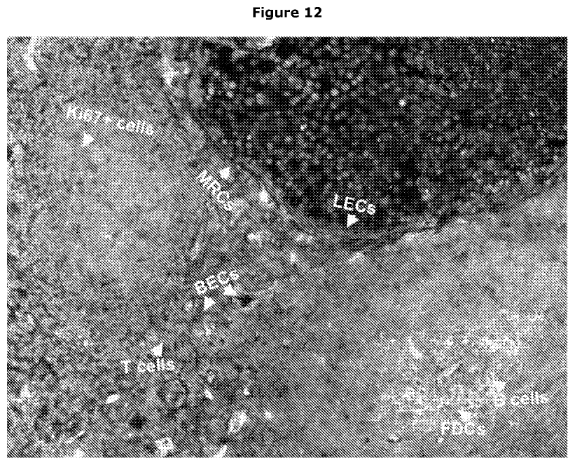

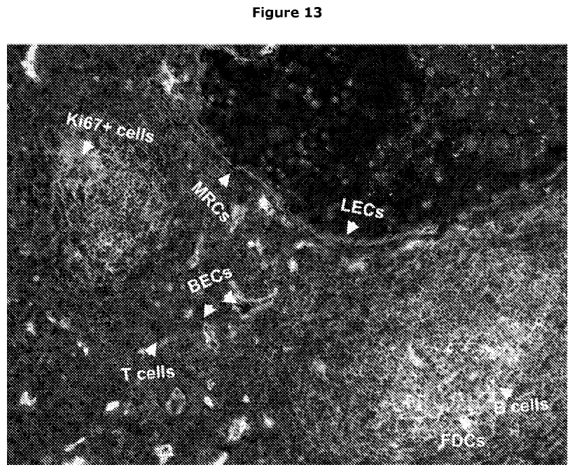

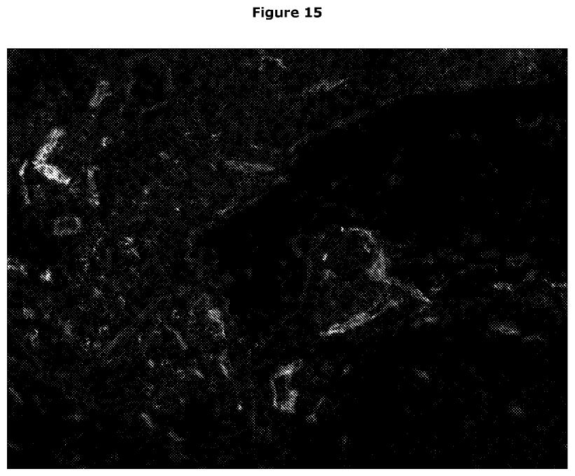

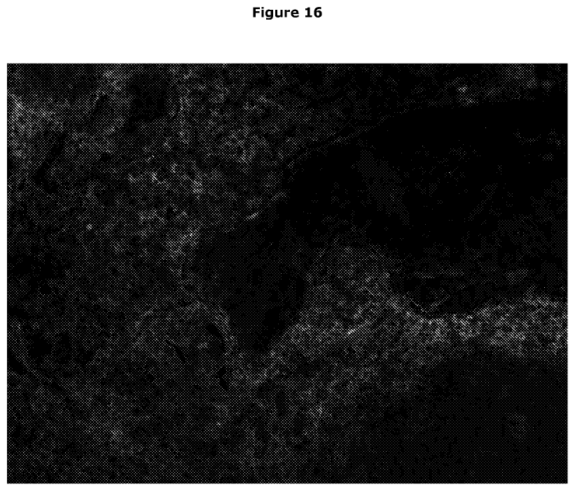

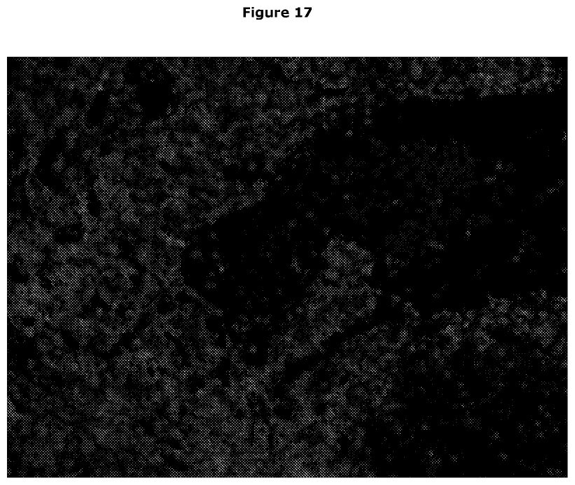

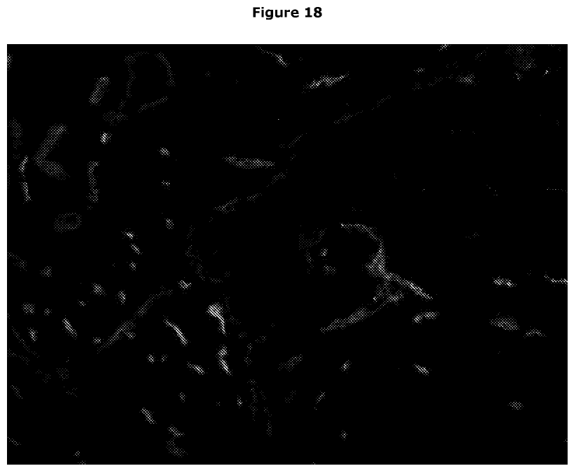

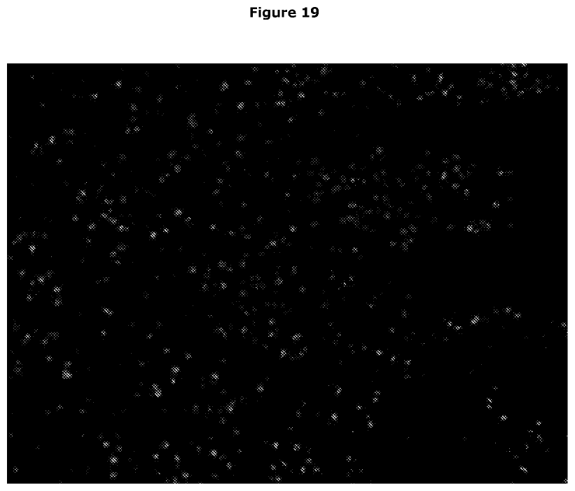

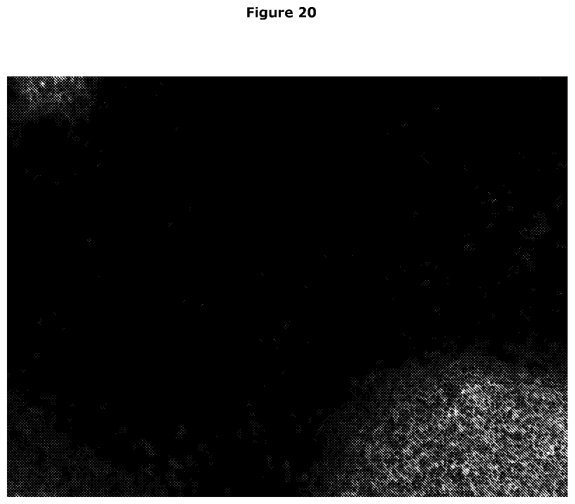

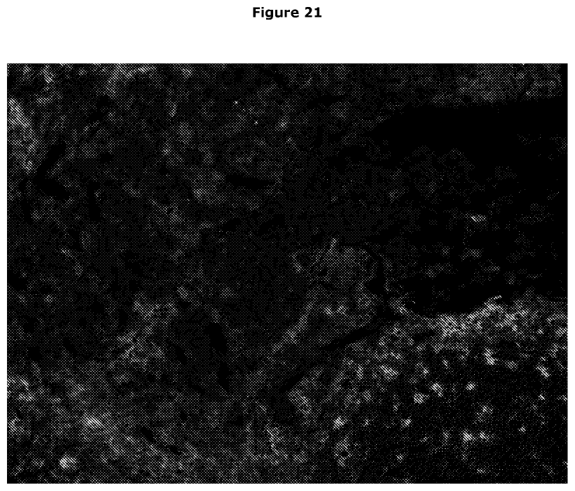

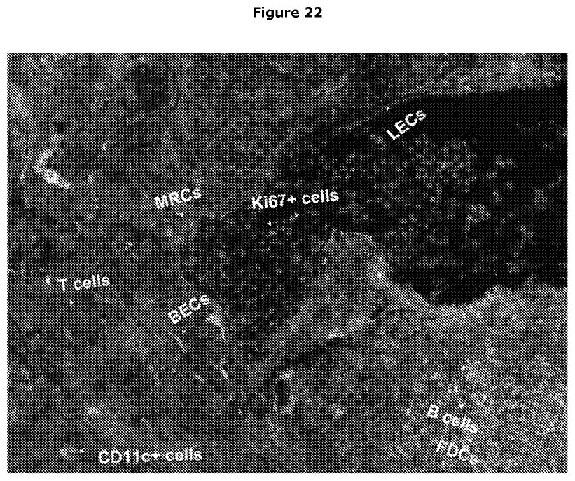

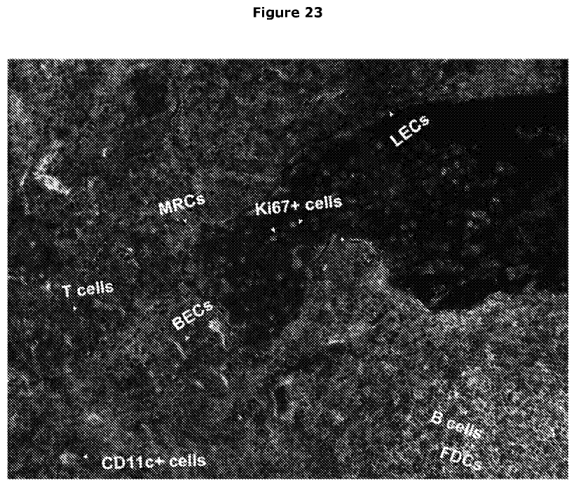

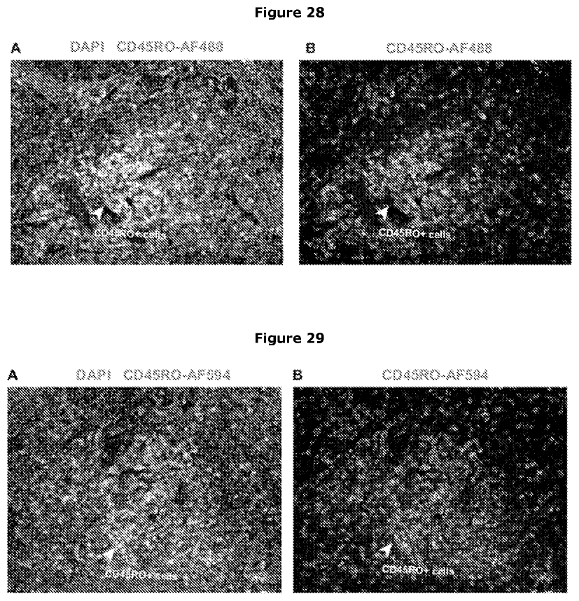

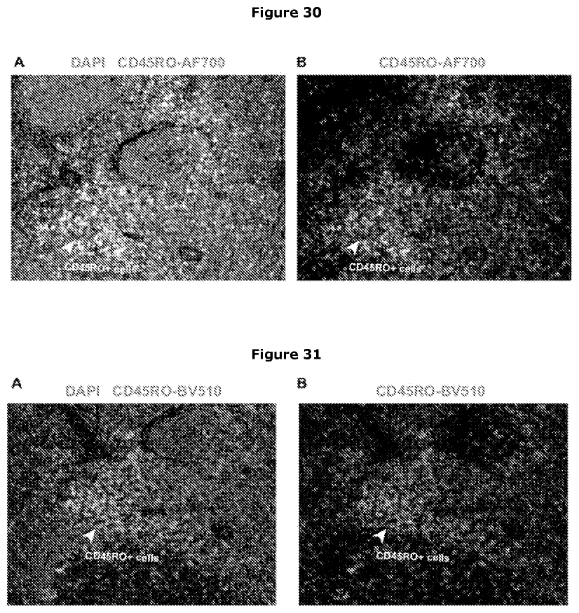

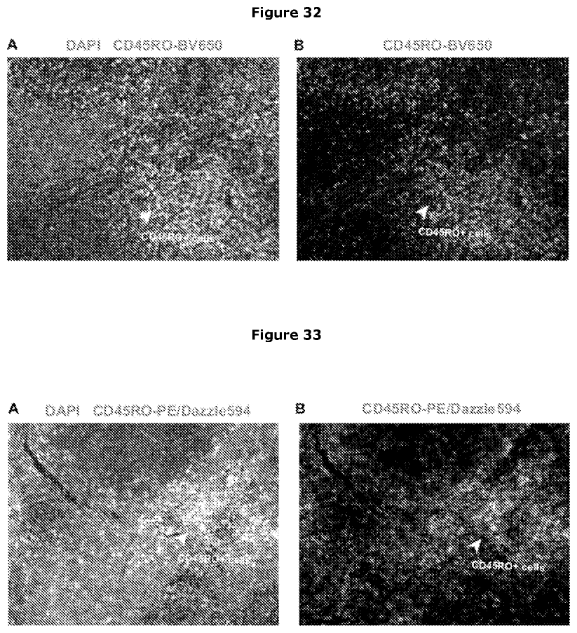

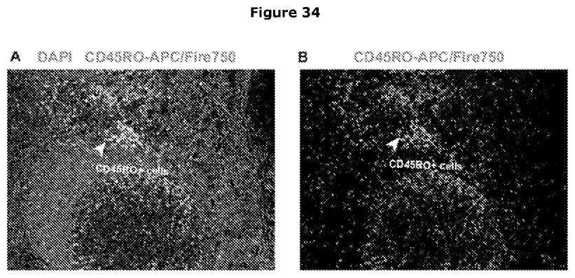

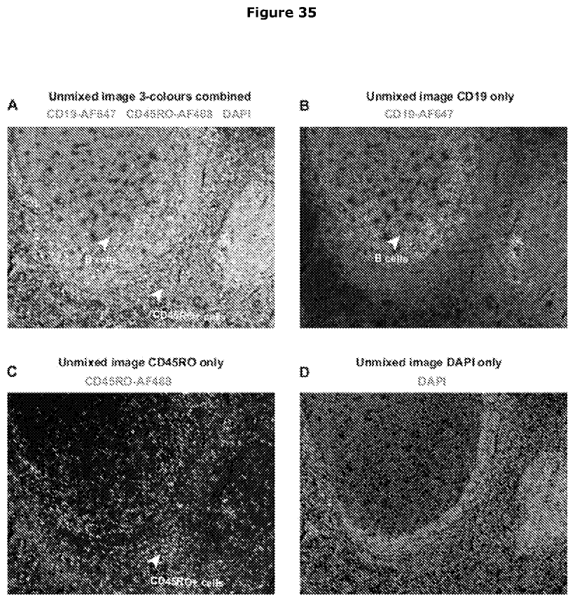

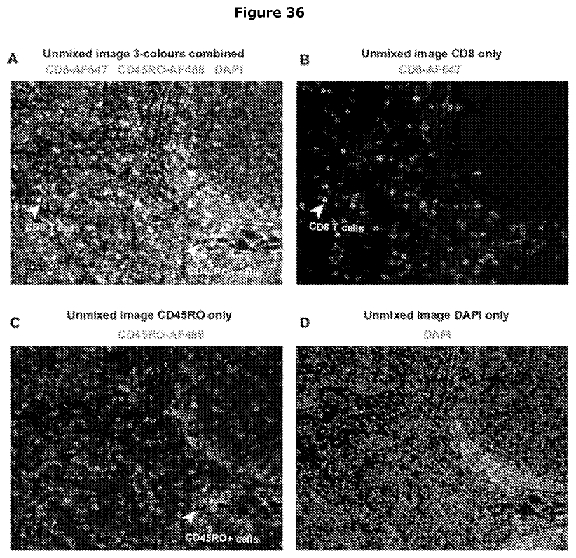

All images in FIGS. 1 to 36 were acquired multispectrally using the Vectra Polaris.

### DETAILED DESCRIPTION OF THE INVENTION

Definitions

Unless otherwise specified, all technical and scientific terms used herein are to be understood as having the same meanings as is understood by one of ordinary skill in the relevant art to which this disclosure pertains. It is also believed that practice of the present invention can be performed using standard immunology, histology, cell biology, molecular biology, pharmacology and biochemistry protocols and procedures as known in the art.

The following definitions are presented to better define the present invention and as a guide for those of ordinary skill in the art in the practice of the present invention.

All patents and publications, including all sequences disclosed within such patents and publications, referred to herein are expressly incorporated by reference.

The term “Ab-FP conjugate” (also abbreviated herein as Ab-FP) and grammatical variations thereof as used herein means an antibody-fluorophore conjugate in which the antibody and the fluorophore in the conjugate are directly linked to each other by at least one covalent bond. In some embodiments an Ab-FP conjugate comprises a single Ab portion having one or more covalently bound FPs. In some embodiments, an Ab-FP comprises a plurality of FPs covalently bound to a single Ab. The antibody in an Ab-FP conjugate is a primary antibody that binds directly to a target antigen that is naturally occurring on a biomolecule.

The term “unique antibody-fluorophore conjugate” including grammatical variations and abbreviations thereof when used in reference to an Ab-FP means that in any composition containing the “unique antibody fluorophore conjugate”, the antibodies and fluorophores comprised in the “unique antibody fluorophore conjugate” are different from any other antibodies or fluorophores comprised in any other Ab-FP comprised in, or that may be used with, the composition. A composition comprising at least two unique Ab-FPs means that the antibodies and the fluorophores in each of the two Ab-FPs are different from each other. Likewise, a composition comprising at least three unique Ab-FPs means that the antibodies and FPs in each of the three Ab-FPs are different from each other.

The terminology “maximum fluorescence excitation and emission wavelength (Ex/Em) and grammatical variations thereof as used herein means the wavelength (nm) of maximum excitation (Ex) of a fluorophore and the wavelength of maximum emission (Em) of that fluorophore.

The terminology “fluorescence excitation and emission spectra” and grammatical variations thereof as used herein refers to a plurality of excitation and emission wavelengths of a given fluorophore that are distinctive for that given fluorophore and that are detected using a multispectral scanner as described herein.

A “multispectral scanner” and grammatical variations thereof as used herein refers to a device that is able to collect data over a variety of different wavelength ranges such as described in U.S. Pat. Nos. 6,399,299 or 9,006,684 which are hereby expressly incorporated by reference including all patents and publications disclosed therein.

The terminology “labeled” and “labeling” and grammatical variations thereof as used herein with reference to an Ab-FP means that the Ab comprised in an Ab-FP has been specially bound to a target antigen in situ in a planar biological sample under conditions that allow immunofluorescence detection of the FP comprised in the Ab-FP.

The term “antibody” and grammatical variations thereof refers to an immunoglobulin molecule having a specific structure that interacts (binds) specifically with a molecule comprising its cognate antigen. In some embodiments the antigen is the antigen that was used for synthesizing the antibody. This antigen is known as the target antigen.

The phrase “each Ab is different from each other Ab” means that each Ab specifically binds a different target antigen.

As used herein, the term “antibody” and grammatical variations thereof broadly includes full length antibodies and may also include certain antigen binding portions and/or fragments thereof. Also included are monoclonal and polyclonal antibodies, multivalent and monovalent antibodies, multi-specific antibodies (for example bi-specific antibodies), chimeric antibodies, human antibodies, humanized antibodies and antibodies that have been affinity matured and antigen binding portions and/or fragments thereof.

A “target antigen” that is “specifically bound” or “specifically labeled” (including grammatical variations thereof) by an antibody in an Ab-FP ad described herein is an antigen that binds preferentially to the target antibody e.g. has less than 25%, or less than 10%, or less than 1% or less than 0.1% cross-reactivity with a non-target antibody. In some embodiments, the target antigen is a protein antigen.

As used herein a “target antigen” means an antigen on a biomolecule in a sample that is directly bound by, and thereby specifically labeled by, the antibody portion of an Ab-FP as described herein. Target antigens as used herein are not primary antibodies to which secondary antibodies can be bound. Such primary antibodies are specifically excluded from being target antigens according to the present invention and as described herein.

Usually, a target antibody will have a binding affinity (dissociation constant (Kd) value), for the antigen or epitope of no more than 10⁻⁶, or 10⁻⁷M, preferably less than about 10⁻⁸M, more preferably less than about 10⁻⁹M, or 10⁻¹⁰, or 10⁻¹¹ or 10⁻¹²M. Binding affinity may be assessed using surface plasma resonance [see, for example U.S. Pat. Nos. 7,531,639 or 6,818,392, each of which is incorporated herein by reference].

The term “cell-type” and grammatical variations thereof as used herein means a group of cells that are defined by the shared presence of one or more expressed target antigens. As used herein a “cell-type” can be any size grouping of cells that express the one or more target antigens, such as a population of cells, a sub-population of cells or a smaller group. By way of non-limiting example, a cell type may be a population of immune cells as known in the art, such as T cells, B-cells, or a sub-population of such cells such as invariant natural killer T cells (iNKT) cells.

The terms “planar sample” and “planar biological sample” and grammatical variations thereof as used herein refer to a substantially planar, i.e., two-dimensional samples of biological material containing cells or any combination of biomolecule complexes, cellular organelles, sub-cellular structures or cellular debris (aka “cellular material”). Planar samples may be obtained by sectioning a three-dimensional sample containing cells or cellular material into sections and mounting the sections onto a planar surface. Planar samples may also be obtained by growing or depositing cells or cellular material on a planar surface, or by adsorbing or absorbing cells or cellular material to a planar surface. In a specific embodiment of the invention, a planar biological sample is a tissue section.

The terminology a “colour” that is “associated with the specific binding of an Ab-FP” and grammatical variations thereof is a colour represented in a fluorescence image that directly correlates with the fluorescence emission spectra of an FP in an Ab-FP conjugate when the Ab-FP is specifically bound to a target antigen in situ in a planar biological sample.

The terminology “a suitable control image” and grammatical variations thereof is well understood by a skilled worker and means an image that has been generated for use as an acceptable comparative control as would be recognized by a person of skill in the art. In one non-limiting example, a suitable control image may be an image of an unlabeled tissue section. In another example, a suitable control image may be an image of a tissue section taken from an earlier time point where progression of a disease or condition is being monitored, or from a healthy individual, or from an individual before disease onset, or after medical treatment, but not limited thereto. It is believed that the generation of a suitable control image may be carried out by a skilled worker with reference to the relevant art in combination with the methods and reagents provided by the present disclosure.

The term “biomarker” and grammatical variations thereof is used herein as understood by the skilled person and encompasses a biomolecule that comprises a target antigen, as well as a cell or cellular structure or cellular sub-structure that comprises the biomolecule. In some embodiments the biomolecule is a protein, a carbohydrate, a lipid or a combination thereof; e.g., a glycolipid or glycoprotein, but not limited thereto. In one embodiment the biomolecule is a protein. In one embodiment the biomolecule is a protein that is expressed on or in a cell.

When used descriptively a “biomarker” means a biomolecule that is known to be associated with and/or indicative of a particular biological process, feature, object, state, status or function. In one non-limiting example a biomarker is indicative of a cell type, a cellular function or a cellular process. In some embodiments, the biomarker is a functional marker, for example a marker of a cellular process that occurs in a number of different cells. In such a case, the relative expression of a biomarker may allow discrimination of different cell types, cellular structures and/or cellular sub-structures by enabling sufficient labeling of the biomarker using an Ab-FP as described herein to determine the presence and/or abundance of the biomarker. In one example a biomarker is associated with a disease state or the status of disease progression, but not limited thereto.

As used herein the phrase “known to be associated with” in reference to a biomarker and grammatical variations thereof means that the biomarker is indicative of a biological feature, molecule, structure, state or status of an organism from which it is measured. In some examples, the presence or absence of a biomarker may be indicative of any one or all of a biological feature, molecule, structure, state or status. In other examples, the absolute or relative abundance of a biomarker may be indicative of any one or all of a biological feature, molecule, structure, state or status.

The terminology “determining the abundance of a cell type”” and grammatical variations thereof means determining the absolute number of cells that comprise a particular biomarker of interest according to the methods as described herein. In some embodiments determining the abundance means identifying the number of cells in a multispectral immunofluorescence image of a sample that have a fluorescence emission intensity that is greater than the predetermined background level of autofluorescence of the sample.

The terminology “determining the relative abundance of a cell type”” and grammatical variations thereof means determining the % number of cells that comprise a particular biomarker of interest from among the total population of that cell type present in a planar sample, again according to the methods as described herein.

The “abundance” means the total number of labeled cells. The relative abundance means the % labeled cells of a given cell type from the total cells of that cell type.

The terminology “multispectral imaging of an entire tissue section” and grammatical variations thereof means that the entire section of the tissue sample that is present on a slide or other carrier supporting the section for labeling and imaging is scanned using a multispectral scanner and that the image created from that scan encompasses the entirety of the tissue section present on the slide or carrier.

The term “patient” as used herein is used interchangeably with “subject” and means the same thing. A patient or subject is an animal. Preferably the animal is a mammal. Preferably the mammal includes human and non-human mammals such as cats, dogs, horses, pigs, cows, sheep, deer, mice, rats, primates (including gorillas, rhesus monkeys and chimpanzees), possums and other domestic farm or zoo animals, but not limited thereto. Preferably, the mammal is human. The term “pre-determined” and grammatical variations thereof when used herein to describe a cell type, disease or condition means that the cell type, disease or condition is known and may be selected by a skilled worker in view of the present disclosure and the art.

The term “clinically relevant levels” and grammatical variations thereof as used herein means that the presence and/or abundance of a biomarker detected according to a method as described herein is recognized as clinically actionable by a skilled worker.

As used herein the terminology “in high abundance” and grammatical variations thereof used to describe a biomarker, cell, cell type, cellular structure or sub-structure means that the abundance or relative abundance of the biomarker, cell, cell type, cellular structure or sub-structure in a sample is recognized by a skilled worker as clinically actionable.

The term “clinically actionable” and grammatical variations thereof as used herein refers to the presence and/or abundance of at least one biomarker in a multispectral immunofluorescence image produced according to a method as described herein, wherein the presence and/or abundance of the detected biomarker provides to the clinician clear and compelling evidence that therapeutic intervention is required.

As used herein the term “unique colour” and grammatical variations thereof means a colour specifically corresponding to the spectra of fluorescence energy emitted from a particular FP in an Ab-FP as described herein.

The term “comprising” as used in this specification and claims means “consisting at least in part of”; that is to say when interpreting statements in this specification and claims which include “comprising”, the features prefaced by this term in each statement all need to be present but other features can also be present. Related terms such as “comprise” and “comprised” are to be interpreted in similar manner.

The term “consisting essentially of” as used herein means the specified materials or steps and those that do not materially affect the basic and novel characteristic(s) of the claimed invention.

The term “consisting of” as used herein means the specified materials or steps of the claimed invention, excluding any element, step, or ingredient not specified in the claim.

It is intended that reference to a range of numbers disclosed herein (for example 1 to 10) also incorporates reference to all related numbers within that range (for example, 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

### DETAILED DESCRIPTION

The basis of the inventive technology described herein is the inventor's determination that multiple target antigens can be labeled simultaneously in a planar biological sample using a plurality of Ab-FP conjugates as described herein. In one example, the labeled sample is a tissue section that can then be imaged using a multispectral scanner and the appropriate image analysis software to generate a multispectral image in which the presence and or abundance of from two to twelve different biomarkers can be determined simultaneously.

The inventors have unexpectedly found that using the Ab-FP conjugates described herein the specific and simultaneous labeling of a plurality of biomarkers can be carried out. In this manner, multispectral images depicting a plurality of colours can be generated, each colour associated with the specific binding of an Ab-FP, and each colour providing specific information related to a particular biomarker.

The ability to simultaneously and rapidly label and directly detect such a large number of biomarkers according to the methods described herein provides the clinician with a powerful research tool having distinct, unanticipated and unexpected advantages in terms of biomarker detection. In addition, the methods described herein can be employed by the skilled worker in various clinical applications including disease diagnosis, patient prognosis and other applications requiring for example, discrimination between multiple cell types in a tissue based on the presence and/or abundance of one or more target antigens/biomarkers.

Biomarkers

A person skilled in the art recognizes that a biomolecular marker or “biomarker” is a term of art describing a biomolecule, in this case a protein, the presence of which may be considered indicative of a particular cell type and/or diagnostic and/or prognostic for a particular biological event or context, such as a disease, cell population, or tissue. Detection of one or more “biomarkers” by various means can be used for a large number of research and clinical purposes.

For example, in the present disclosure, a biomarker may be any target biomolecule, preferably a protein, wherein the target biomolecule is detected and visualized according to the multispectral immunofluorescence detection methods described herein by specific binding of an Ab-FP as described herein to a target antigen comprised on the biomolecule. The target antigen is present on the biomolecule which itself will be present in or on a cell. In this fashion a biomarker may be used to detect a cell, cell type, cellular structure or sub-structure, but not limited thereto. In one embodiment, the biomolecule is a protein or antigen comprising portion thereof.

In one exemplary situation, a target antigen on an CD21 protein is labeled using an Ab-FP conjugates as described herein, wherein the target antigen is present on a protein, wherein that protein is present on a cell and serves as one of several biomarkers of mature-B cells and follicular dendritic cells (but not limited thereto).

Multispectral immunofluorescence detection of a plurality of biomarkers for a number of different research, diagnostic and prognostic purposes is specifically contemplated as part of the methods described herein. The inventors believe that in view of the present disclosure, a skilled worker using the methods of the invention as described herein can simultaneously detect the presence and abundance of a plurality of cells or cell types as follows.

The skilled worker can select an appropriate set of at least three, preferably four, five, six, preferably at least seven antibodies that specifically bind to at least three, preferably four, five, six, preferably at least seven target antigens, each comprised on a pre-determined biomarker respectively. The selected antibodies are then directly conjugated to one of the FPs as described herein to form an Ab-FP conjugate as described herein for simultaneous multispectral imaging of the biomarkers.

In this manner the methods described herein can be employed by the skilled person to carry out research on various cells, cell populations and tissues, as well as to perform diagnoses and/or prognoses of many different diseases or conditions. In some embodiments the diseases or conditions are immunological diseases or conditions. The methods described herein can also be employed clinically to identify patient populations and sub-populations by immuno-populations and sub-populations of cells, and to monitor the effects of drugs and/or candidate drugs on target antigen expression, but not limited thereto.

By way of non-limiting example, immune-populations of cells of relevance for identification in a clinical setting include CD4+ T cells including regulatory T cells, CD8+ T cells (cytotoxic), B cells (including naïve, activated, memory and plasma cells), monocytes, dendritic cells, macrophages and tumour cells.

The inventors believe that the advantages of the methods and reagents provided herein are numerous when viewed in comparison to methods available in the art.

Traditional microscope-based immunofluorescence microscopy (IFM) has been restricted to the detection of four different labels, each corresponding to a colour that itself corresponds to the fluorescence emission spectra of a different fluorophore comprised in a label. The use of a nuclear stain such as DAPI or HOECHST 33342 meant that the skilled practitioner could visualize up to three proteins and a nuclear stain simultaneously within a single field of view (e.g., by rapidly exchanging filter sets).

IFM in clinical pathology is typically employed to generate single colour images only such as to detect antibody deposits in organs like the kidney. Instead, when visualization of a biomarker is required, a clinical pathologist will typically use enzymatic immunohistochemistry (IHC). Although two and even three-colour methods are available for immunohistochemical staining, the vast majority of clinical pathology laboratories use a single colour only, with typical visualization being based on the binding of horseradish peroxidase-labeled secondary antibodies. Quantification of cells is generally done manually. Manual visualization can be difficult and is subject to inaccuracy and error, relying on interpretation in many instances such as when visualizing cancer cells. A skilled worker has to rely on the size and shape of the cell to identify a cancer cell, and then count how many of the identified cells are labeled. This process is known to be fraught with inaccuracy.

Multiplexed IFM methods that are currently employed typically permit the identification of up to four proteins at the same time. Traditional multiplexed IFM methods (with formalin fixed paraffin embedded (FFPE) tissues) are usually performed in two to four colours due to the following reasons: 1) limited availability of primary antibodies in different isotypes (if from the same host animal) and host species, 2) limited availability of fluorophores that can be well separated by conventional IF microscope filters.

Current applications using multispectral imaging have increased the number of colours that can be visualized, as well as the field of view that can be analyzed. Specifically, multispectral scanning can be used to generate a multispectral image comprising a plurality of colours, each colour corresponding to the fluorescence emission spectra of a different fluorophore. Visualization of different emission spectra across an entire sample multispectrally can be done in a single scan.

However, there are a number of recognized disadvantages attached to the methods currently employed for multiplex labeling of tissue sections for multispectral imaging including the great length of time required for preparation of samples for multiplex imaging, a lack of reproducibility and the great expense.

For example, certain iterative labeling protocols can offer a large number of different colours (>40 colours); e.g., the CODEX system (https://www.akoyabio.com/codextm/technology). The CODEX system is an IFM based platform that uses an iterative labeling protocol employing oligonucleotide-conjugated Abs, added at three per antibody binding cycle, followed by repeated stripping and rebinding of antibodies to create a multiplex labeled sample. At present there are only a limited number of oligonucleotide-conjugated antibodies available. These antibodies are specialized expensive labeled antibodies, not off-the-shelf FP-conjugated antibodies. CODEX can be equipped with a multispectral scanner, but multispectral imaging using CODEX is limited to three colours. Modification of existing labeling protocols, and production of new protocols and labeling panels for this platform is slow due to the iterative labeling process.

In CODEX, generation of tissue samples that allows such imaging is not done by simultaneous labeling of a tissue section with a plurality of Ab-FPs as described herein. Rather, this technique requires the tedious staining and stripping protocols referred to above and employs secondary fluorescence labeling via oligonucleotide tags.

In contrast, the methods described herein are distinctly advantageous for the clinician in enabling equivalent staining protocols to be carried out in as little as two hours and iterated within a matter of days. These advantages are facilitated by the speed of the multispectral scan employed in the methods as described herein. In some embodiments generating a multispectral fluorescence image of a sample is done in less than 10 min, less than 20 min, preferably less than 30 min.

Examples of multiplex immunofluorescence imaging methods currently available include MACSima™ (Miltenyi Biotec), and Chip-cytometry Zellscanner one (Zellkraftwerk). However, each of these technologies suffers from several disadvantages as discussed herein.

MACSima and Zellscanner also require iterative labeling. The MACSima system allows for only three antibodies at a time. The MACSima system is equipped with a conventional fluorescence microscope and does not allow for multispectral imaging of an entire tissue section.

The Zellscanner system (http://www.zellkraftwerk.com/products) allows for the addition of up to five specific fluorophore-conjugated Abs per cycle (BUV395, BUV421, FITC, PE, PerCP) and repeat labeling cycles. However, Zellscanner is limited to the use of these five fluorophores, as the instrument is specifically designed for these fluorophores alone. The Zellscanner is equipped with a conventional fluorescence microscope and is not capable of multispectral scanning. An additional major drawback of this system is that the tissue section to be examined must be loaded onto a special chip to be examined by the instrument. This requirement makes the use of Zellscanner difficult with tissue samples that are already frozen, or paraffin embedded.

Again, modification of existing labeling protocols, and production of new protocols and labeling panels for both MACSima and Zellscanner platforms is slow due to the iterative labeling process.

Ab-FP Labeling of Formalin Fixed Paraffin Embedded (FFPE) Tissue

Antibody labeling of FFPE tissue using traditional multiplexed IF methods (i.e. indirect IF with primary antibodies against targets and fluorophore-conjugated secondary antibodies that bind primary antibodies) is mostly performed in two to four colours, due to the 1) limited availability of primary antibodies in different isotypes (if from the same host animal) and host species, 2) limited availability of fluorophores that can be well separated by conventional IF microscope filters.

Staining FFPE tissue IF in two to four colours using a traditional multiplexed IFM method typically takes—one and a half to two days.

New Opal IHC technique allows multiplexed IF staining of FFPE tissue up to seven to nine colours (provided that a microscope capable of multispectral imaging is available). However, Opal staining takes a minimum of three to four days to complete the entire staining cycle. Furthermore, developing and optimizing a seven colour Opal staining panel can take six to eight weeks (please see this webpage for more information: https://www.akoyabio.com/product-support/opal-multiplex-immunohistochennistry#Opal-FAQ), and users require considerable knowledge and experience of IHC techniques to design and develop Opal staining panels. Additionally, the Opal platform is not compatible with frozen tissue sections.

Thus, because multiplex staining using the Opal multiplex IHC technique is slow, technically difficult due to the large number of steps, and can be used with only a limited number of staining reagents, this technique is not widely used in clinical diagnostics and research labs.

In providing the methods and reagents as described herein the inventors have overcome a number of the disadvantages set forth above by direct labeling of target antigens using the directly conjugated Ab-FP conjugates as described herein in methods of simultaneous labeling and detection of biomarkers.

The methods described herein are not sandwich assays and do not encompass primary labeling of a target antigen with a first antibody that specifically binds a target antigen, followed by secondary labeling of the first antibody with an antibody-fluorophore conjugate, the fluorophore then being detected by fluorescence emission. Rather, the methods described require the Ab-FPs as described herein, wherein the antibody portion of the Ab-FP is a primary antibody that specifically binds the target antigen and the FP portion is directly linked to the primary Ab. A skilled worker in the art appreciates that following the methods described herein provides the advantage of direct detection of a target antigen by an Ab-FP as described herein without the need for secondary labeling as employed in sandwich assays as known in the art.

A major advantage provided by the present invention is the use of antibodies directly conjugated to fluorophores (i.e., directly conjugated antibodies) to rapidly allow the multispectral detection and imaging of an entire slide/section. In some embodiments the advantages also encompass the use of a plurality of directly conjugated antibodies to rapidly and simultaneously allow the multispectral detection and imaging of an entire slide/section. In one non-limiting example, this is done using the Vectra Polaris.

The inventors believe that they are the first to provide a skilled worker with the ability to rapidly generate new diagnostic tests that visualize all the clinically relevant cellular markers in a single image, enabling quantification of relative expression in different cells. For example, following the methods described herein a multiplex fluorescence image of a planar biological sample comprising at least three and up to twelve target antigens can be generated in less than fifteen minutes. By these means, the inventors have provided a surprising and effective technical solution that will rapidly open up new opportunities in cellular and tissue research as well as in clinical medicine. The ability to identify cells and/or cell populations, and/or to carry out such diagnostic tests within hours as provided herein offers clear and distinct advantages over existing technologies and offers the skilled worker the means to improve the efficiency and accuracy of both research and diagnostic pathological applications in many fields.

The inventors further believe that the skilled person readily appreciates that the methods and reagents described herein provide a non-obvious technical solution that enables the development of new applications in biomedical research and medicine as described herein. The methods and reagents described herein further enable the development of rapid diagnostics that can be employed to accelerate the selection of optimal therapeutic regimen for patients. For example, in some embodiments the methods disclosed herein can be employed by the skilled person for molecular and immune profiling and disease management of cancer, including lung, breast and colorectal cancers by choice of the appropriate plurality of biomarkers (for example with reference to (Hofman, 2019) (Sood, 2016) and (Majtahed, 2011), the disclosures of which are all expressly incorporated herein by reference in their entireties).

Accordingly, in one aspect the present invention relates to a composition comprising at least three, four, five, six or at least seven antibody-fluorophore conjugates (Ab-FP), wherein each FP has a different fluorescence excitation and emission spectra (Ex).

In one embodiment each Ab is different from each other Ab.

In one embodiment, the composition comprises at least eight, nine, ten, eleven or twelve Ab-FPs. In one embodiment, the composition comprises six, seven or eight Ab-FPs.

In one embodiment the composition consists essentially of at least three, four, five, six, seven, eight, nine, ten, eleven, or twelve Ab-FPs. In one embodiment the composition consists essentially of at least three to at least seven Ab-FPs. In one embodiment the composition consists essentially of at least six, seven, or eight Ab-FPs.

In one embodiment the composition consists essentially of at least six Ab-FPs. In one embodiment the composition consists essentially of at least seven Ab-FPs. In one embodiment the composition consists essentially of at least eight Ab-FPs.

In one embodiment the composition consists essentially of six Ab-FPs. In one embodiment the composition consists essentially of seven Ab-FPs. In one embodiment the composition consists essentially of eight Ab-FPs.

In one embodiment each Ab-FP in the composition is a unique Ab-FP.

In one embodiment each FP has a maximum excitation and emission wavelength (Ex/Em) selected from the group consisting of 348/395, 404/448, 405/421, 405/510, 405/570, 405/603, 405/646, 405/711, 407/421, 415/500, 436/478 nm, 490/515 nm, 494/520 nm, 495/519 nm, 485/693 nm, 496/578, 532/554 nm, 566/610 nm, 590/620 nm, 650/660 nm, 650/668 nm, 652/704, 696/719 nm, 753/785 nm, 754/787 nm, 755/775 nm and 759/775 nm.

In one embodiment the maximum fluorescence emission wavelength (Em) of at least one, two or three of the FPs is about 710 nm to about 850 nm. In one embodiment the Em of at least one, two or three of the FPs is about 753 nm to about 759 nm, preferably of 753 nm, 754 nm, 755 nm or 759 nm. In one embodiment the Em of one of the FPs is 754 nm.

In one embodiment the FP is selected from the group consisting of Brilliant™ Ultraviolet 395 (BUV395) having an Ex/Em of 348/395, Brilliant™ Violet 480 (BV480) having an Ex/Em of 436/478 nm, Brilliant Violet 421™ having an Ex/Em of 405/421, Brilliant™ Violet 421 (BV421) having an Ex/Em of 407/421, Brilliant™ Violet 510 (BV510) having an Ex/Em of 405/510, Brilliant Violet 570™ having an Ex/Em of 405/570, Brilliant Violet 605™ having an Ex/Em of 405/603, Brilliant Violet 650™ having an Ex/Em of 405/646, Brilliant Violet 711™ having an Ex/Em of 405/711, BD Horizon™ V450 having an Ex/Em of 404/448, BD Horizon™ V500 having an Ex/Em of 415/500, Brilliant™ Blue 515 (BB515) having an Ex/Em of 490/515 nm, Fluorescein Isothiocyanate (FITC) having an Ex/Em of 494/520 nm, Alexa Fluor 488 (AF488) having an Ex/Em of 495/519 nm, Alexa Fluor 532 (AF532) having an Ex/Em of 532/554 nm, R-phycoerythrin (PE) having an Ex/Em of 496/578, Alexa Fluor 594 (AF594) having an Ex/Em of 590/620 nm, PE-Dazzle 594 (PE594) or PE-CF594 (CF594) having an Ex/Em of 566/610 nm, Alexa Fluor 647 (AF647) having an Ex/Em of 650/668 nm, Allophycocyanin (APC) having an Ex/Em of 650/660, BD Horizon™ 700 (BB700) having an Ex/Em of 485/693 nm, Alexa Fluor 700 (AF700) having an Ex/Em of 696/719 nm, APC/Alexa Fluor 750 having an Ex/Em of 753/785 nm, APC/Fire 750 having an Ex/Em of 754/787 nm, APC-R700 having an Ex/Em of 652/704, APC-Cy7 having an Ex/Em of 755/775 nm and AF750 having an Ex/Em of 759/775 nm.

In one embodiment the fluorophore is selected from the group consisting of BV480, BB515, AF532, PE-CF594, AF647, and AF700 or BB700.

In one embodiment the fluorophore is selected from the group consisting of BV480, BB515, AF532, PE-CF594, AF647, AF700 and BB700.

In one embodiment the fluorophore is selected from the group consisting of AF647, AF488, AF594, AF700, BV510, BV650, PE/Dazzle594 and APC/Fire750.

In one embodiment the Ab in the Ab-FP is selected from the group consisting of anti-CD31, CD141, CD144, CD3, CD34, CD163, CD11c, CD14, CD16, CD68 Foxp3, CD4, CD8, CD19 CD20, CD25, CD45RO, CD45RA, CD38, PD-1, PDL1, PDL2, CD68, Ki-67, Sox10, S100, PRAME, MART1 and anti-CD21 antibodies.

In one embodiment the Ab in the Ab-FP is selected from the group consisting of CD19, CD8 and CD45RO.

In one embodiment the Ab in the Ab-FP comprises one or more Ab selected from the group consisting of anti-oestrogen receptor (ER), progesterone receptors (PR), her2 and anti-cytokeratin antibodies.

In one embodiment the Ab in the Ab-FP comprises one or more anti-mismatch repair protein antibodies or anti-mutant mismatch repair protein antibodies.

In one embodiment the Ab in the Ab-FP comprises one or more antibodies selected from the group consisting of anti-b-raf (V600E mutation), MLH1, MSH2, MSH6, and anti-PMS2 antibodies.

In one embodiment the Ab in the Ab-FP is selected from the group consisting of anti-CD31, CD141, CD3, CD34, Ki-67, CD11c, and anti-CD21 antibodies.

In one embodiment the composition comprises at least three, preferably four, five, or preferably all six of the following antibodies: anti-CD31, CD141, CD3, CD34, Ki-67, and anti-CD21 antibodies.

In one embodiment the composition comprises three, preferably four, five, or preferably all six of the following antibodies: anti-CD31, CD141, CD3, CD34, Ki-67, and anti-CD21 antibodies.

In one embodiment the composition comprises at least one, preferably at least two, of the following antibodies: anti-CD19, CD8 and anti-CD45RO antibodies.

In one embodiment the composition comprises one, preferably two, of the following antibodies: anti-CD19, CD8 and anti-CD45RO antibodies.

In one embodiment the composition comprises at least three, preferably four, five, or preferably all six of the following Ab-FPs: CD31-BV480; CD141-BB515; CD3-AF532; CD34-PE-CF594; Ki67-AF647 and CD21-BB700.

In one embodiment the composition comprises three, preferably four, five, or preferably all six of the following Ab-FPs: CD31-BV480; CD141-BB515; CD3-AF532; CD34-PE-CF594; Ki67-AF647 and CD21-BB700.

In one embodiment the composition comprises at least three, preferably four, five, six or preferably all seven of the following antibodies: anti-CD31, CD141, CD3, CD34, Ki-67, CD11c and anti-CD21 antibodies.

In one embodiment the composition comprises three, preferably four, five, six or preferably all seven of the following antibodies: anti-CD31, CD141, CD3, CD34, Ki-67, CD11c and anti-CD21 antibodies.

In one embodiment the composition comprises at least three, preferably four, five, six or preferably all seven of the following Ab-FPs: CD31-BV480; CD141-BB515; CD3-AF532; CD34-PE-CF594; Ki67-AF647, CD11c-AF700 and CD21-BB700.

In one embodiment the composition comprises three, preferably four, five, six or preferably all seven of the following Ab-FPs: CD31-BV480; CD141-BB515; CD3-AF532; CD34-PE-CF594; Ki67-AF647, CD11c-AF700 and CD21-BB700.

In one embodiment the composition comprises at least one, preferably at least two, of the following Ab-FPs: CD19-AF647; CD8-AF647; CD45RO-AF488; CD45RO-AF594; CD45RO-AF700; CD45RO-BV510; CD45RO-BV650; CD45RO-PE/Dazzle594 and CD45RO-APC/Fire750.

In one embodiment the composition comprises one, preferably two, of the following Ab-FPs: CD19-AF647; CD8-AF647; CD45RO-AF488; CD45RO-AF594; CD45RO-AF700; CD45RO-BV510; CD45RO-BV650; CD45RO-PE/Dazzle594 and CD45RO-APC/Fire750.

In one embodiment each Ab specifically binds a target antigen. In one embodiment each Ab specifically binds a different target antigen. In one embodiment each target antigen is a biomarker for a protein or cell type. In one embodiment each target antigen is a biomarker for a different protein or different cell type.

In one embodiment each target antigen is a biomarker for a cell surface receptor or an intracellular antigen. In one embodiment the intracellular antigen is a nuclear antigen.

In one embodiment each target antigen is a T cell, B cell, macrophage, monocyte or dendritic cell antigen.

In one embodiment each target antigen is a T cell antigen selected from the group consisting of CD3, CD4, CD8, FoxP3, CD25, CD137, CD38, CD69, PD-1, CTLA-4, CD45RO, CD45RA and Ki67 antigens.

In one embodiment each target antigen is a B cell antigen selected from the group consisting of CD19, CD20, CD21, BCL-6, BLIMP1, Ki67 and CD138 antigens.

In one embodiment each target antigen is a macrophage or monocyte antigen selected from the group consisting of CD14, CD16, CD68, and CD163 antigens.

In one embodiment each target antigen is a dendritic cell antigen that is CD1c or CLEC9a antigens.

In one embodiment the target antigens are biomarkers associated with a disease or condition.

In one embodiment the biomarker or biomarkers are diagnostic of a disease or condition.

In one embodiment the biomarker or biomarkers are prognostic for a disease or condition. In one embodiment the disease or condition is cancer, preferably breast cancer, lung cancer, colorectal cancer or melanoma. In one embodiment the disease or condition is an immunological disease or condition.

In another aspect the invention relates to a method of direct immunofluorescence analysis of biological sample comprising
a) labeling at least one target antigen in a planar biological sample with at least one unique Ab-FP conjugate, and
b) generating a multispectral fluorescence image of the labeled planar sample using a multispectral scanner, wherein the image comprises at least two colours, wherein at least one colour is associated with the specific binding of the at least one unique Ab-FP conjugate to the at least one target antigen, and
c) determining from the image the presence or absence of at least one biomarker comprising the at least one target antigen.

In one embodiment labeling in a) comprises simultaneously labeling at least two, preferably at least three, four, five, six, preferably at least seven different target antigens with at least two, preferably at least three, four, five, six, preferably at least seven unique Ab-FP conjugates.

In one embodiment labeling in a) comprises simultaneously labeling two, preferably three, four, five, six, preferably seven different target antigens with two, preferably three, four, five, six, preferably seven unique Ab-FP conjugates.

In one embodiment, one colour in b) is associated with the fluorescence emission spectra of a nuclear stain. In one embodiment the nuclear stain is DAPI or Hoechst 33342.

In one embodiment the multispectral image in b) comprises at least three, preferably at least four, five, six, preferably seven different colours, wherein each different colour is associated with the specific binding of an Ab-FP to a target antigen.

In one embodiment the multispectral image in b) comprises three, preferably at least four, five, six, preferably seven different colours, wherein each different colour is associated with the specific binding of an Ab-FP to a target antigen.

In one embodiment generating the multispectral image in b) comprises detecting at least two, preferably three, four, five, six, preferably seven different fluorescence spectra respectively using the multispectral scanner, wherein each spectrum detected corresponds to an Ab-FP that is specifically bound to a target antigen.

In one embodiment generating the multispectral image in b) comprises detecting two, preferably three, four, five, six, preferably seven different fluorescence spectra respectively using the multispectral scanner, wherein each spectrum detected corresponds to an Ab-FP that is specifically bound to a target antigen.

In one embodiment the planar biological sample is a tissue section. In one embodiment the tissue section is a frozen or formalin fixed tissue section. In one embodiment the tissue section is a frozen tissue section. In one embodiment the tissue section is a formalin fixed paraffin embedded tissue section.

In one embodiment the tissue section is from a mammal, preferably a human.

In one embodiment the tissue section is from a liver, lung, breast, colon, tonsil or a lymph node. In one embodiment the tissue section is from a biopsy of a tissue having or suspected of having at least one cancerous cell. In one embodiment the tissue biopsy is from a liver, lung, breast, colon or a lymph node. In one embodiment the tissue biopsy is a cancer biopsy. In one embodiment the tissue biopsy is a tumour biopsy.

In one embodiment the at least one target antigen is a biomarker for a biomolecule. In one embodiment the biomolecule is or comprises a protein, a carbohydrate or a lipid or an antigenic portion thereof. In one embodiment the biomolecule is a protein or antigenic portion thereof. In one embodiment the protein is expressed in or on a cell, cellular structure or cellular sub-structure.

In one embodiment the at least one target antigen is a biomarker for a cell, cell type, cellular structure or cellular sub-structure. In one embodiment the presence of a biomarker defines a cell type. In one embodiment the presence of one or more biomarkers defines a cell type.

In one embodiment the abundance of a biomarker defines a cell type. In one embodiment the abundance of one or more biomarkers defines a cell type. In one embodiment the relative abundance of a biomarker defines a cell type. In one embodiment the relative abundance of one or more biomarkers defines a cell type.

In one embodiment the biomolecule is a biomarker for a cell surface receptor or an intracellular antigen. In one embodiment the intracellular antigen is a nuclear antigen.

In one embodiment the at least one target antigen is a biomarker for a T cell, B cell, macrophage, monocyte or dendritic cell.

In one embodiment at least one target antigen is selected from the group consisting of anti-oestrogen receptor (ER), progesterone receptors (PR), her2, and anti-cytokeratin antigens.

In one embodiment at least one target antigen is a mismatch repair protein or a mutant mismatch repair protein antigen.

In one embodiment at least one target antigen is selected from the group consisting of b-raf (V600E mutation), MLH1, MSH2, MSH6, and PMS2 antigens.

In one embodiment the Ab in the Ab-FP is selected from the group consisting of anti-CD31, CD141, CD3, CD34, Ki-67, CD11c, and anti-CD21 antibodies.

In one embodiment the at least one target antigen is a T cell antigen selected from the group consisting of CD3, CD4, CD8, FoxP3, CD25, CD137, CD38, CD69, PD-1, CTLA-4, CD45RO, CD45RA and Ki67 antigens.

In one embodiment the at least one target antigen is a B cell antigen selected from the group consisting of CD19, CD20, CD21, BCL-6, BLIMP1, Ki-67 and CD138 B cells antigens.

In one embodiment each target antigen is a macrophage or monocyte antigen selected from the group consisting of CD14, CD16, CD68, and CD163 antigens.

In one embodiment the at least one target antigen is the dendritic cell antigen CD1c or CLEC9a antigens.

In one embodiment the at least one target antigen is a biomarker associated with a disease or condition. In one embodiment the biomarker is diagnostic or partially diagnostic of a disease or condition. In one embodiment the biomarker is prognostic or partially prognostic for a disease or condition. In one embodiment the disease or condition is cancer, preferably breast, lung or colorectal cancer or melanoma.

In one embodiment the biomarker discriminates or partially discriminates between patient populations.

In one embodiment the biomarker discriminates or partially discriminates between patients who have responded to a particular therapy and those who have not.

In one embodiment the biomarker discriminates or partially discriminates between patients who are more likely to be responsive to a particular therapy, and those that are less likely to be responsive.

In one embodiment the therapy is an immune therapy. In one embodiment the therapy is a cancer therapy.

In one embodiment the Ab in the Ab-FP is selected from the group consisting of anti-CD31, CD141, CD3, CD34, CD163, CD11c, Cd14, CD16, Foxp3, CD4, CD8, CD19, CD20, CD25, CD38, CD45RO, CD45RA, PD-1, PDL1, PDL2, CD68, CD14, Ki-67, Sox10, S100, PRAME, MART1 and anti-CD21 antibodies.

In one embodiment the Ab in the Ab-FP comprises one or more antibodies selected from the group consisting of anti-oestrogen receptor (ER), progesterone receptors (PR), her2, and anti-cytokeratin antibodies.

In one embodiment the Ab in the Ab-FP comprises one or more anti-mismatch repair protein or mutant mismatch repair protein antibody.

In one embodiment the Ab in the Ab-FP comprises one or more antibodies selected from the group consisting of anti-b-raf (V600E mutation), MLH1, MSH2, MSH6, and anti-PMS2 antibodies.

In one embodiment the Ab in the Ab-FP is selected from the group consisting of anti-CD31, CD141, CD3, CD34, Ki-67, CD11c and anti-CD21 antibodies.

In one embodiment the Ab in the Ab-FP is selected from the group consisting of CD19, CD8 and CD45RO.

In one embodiment labeling in a) comprises simultaneously labeling with two to seven unique Ab-FPs, preferably with six unique Ab-FPs, preferably with seven unique Ab-FPs.

In one embodiment labeling in a) further comprises simultaneously labeling with at least two Ab-FPs, preferably with at least three, four, five or six additional unique Ab-FPs.

In one embodiment labeling in a) comprises labeling with at least two, preferably three, four, five, six or preferably all seven of the following antibodies: anti-CD31, CD141, CD3, CD34, Ki67, CD11c and anti-CD21 antibodies.

In one embodiment labeling in a) comprises labeling with two, preferably three, four, five, six or preferably all seven of the following antibodies: anti-CD31, CD141, CD3, CD34, Ki67, CD11c and anti-CD21 antibodies.

In one embodiment labeling in a) comprises labeling with at least two, preferably three, four, five, six or preferably all seven of the following Ab-FPs: CD31-BV480; CD141-BB515; CD3-AF532; CD34-PE-CF594; Ki67-AF647; CD11c-AF700 and CD21-BB700.

In one embodiment labeling in a) comprises labeling with two, preferably three, four, five, six or preferably all seven of the following Ab-FPs: CD31-BV480; CD141-BB515; CD3-AF532; CD34-PE-CF594; Ki67-AF647; CD11c-AF700 and CD21-BB700.

In one embodiment labeling in a) comprises labeling with at least one, preferably at least two of the following antibodies: anti-CD19, CD8 and anti-CD45RO antibodies.

In one embodiment labeling in a) comprises labeling with one, preferably two of the following antibodies: anti-CD19, CD8 and anti-CD45RO antibodies.

In one embodiment labeling in a) comprises labeling with at least one, preferably at least two, of the following Ab-FPs: CD19-AF647; CD8-AF647; CD45RO-AF488; CD45RO-AF594; CD45RO-AF700; CD45RO-BV510; CD45RO-BV650; CD45RO-PE/Dazzle594 and CD45RO-APC/Fire750.

In one embodiment labeling in a) comprises labeling with one, preferably two, of the following Ab-FPs: CD19-AF647; CD8-AF647; CD45RO-AF488; CD45RO-AF594; CD45RO-AF700; CD45RO-BV510; CD45RO-BV650; CD45RO-PE/Dazzle594 and CD45RO-APC/Fire750.

In one embodiment labeling in a) further comprises replacing one Ab-FP with a different Ab-FP. In one embodiment the replacement Ab-FP comprises the same Ab and an FP having an Ex/Em of about 753 nm to about 759 nm, preferably of 753 nm, 754 nm, 755 nm or 759 nm.

In one embodiment labeling in a) further comprises labeling an additional target antigen with an additional Ab-FP comprising an FP having an Ex/Em of about 753 nm to about 759 nm, preferably of 753 nm, 754 nm, 755 nm or 759 nm.

In one embodiment the additional Ab-FP comprises an anti-CD31, CD141, CD3, CD34, Ki-67, CD11c or anti-CD21 Ab. In one embodiment the additional Ab does not comprise an anti-CD31, CD141, CD3, CD34, Ki-67, CD11c or anti-CD21 Ab.

In one embodiment determining in c) is detecting the presence of at least two, preferably at least three, four, five, six, preferably at least seven different biomarkers.

In one embodiment determining in c) is detecting the presence of two, preferably at least three, four, five, six, preferably at least seven different biomarkers.

In one embodiment determining in c) comprises determining the abundance of the at least two, preferably at least three, four, five, six, preferably at least seven different biomarkers. In one embodiment determining the abundance is determining the relative abundance of each different biomarker.

In one embodiment determining in c) comprises determining the abundance of the two, preferably at least three, four, five, six, preferably at least seven different biomarkers. In one embodiment determining the abundance is determining the relative abundance of each different biomarker.

In one embodiment determining the abundance is determining in c) comprises determining the number of cells in the image that comprise one or more labeled biomarkers.

In one embodiment determining the relative abundance comprises determining in c) the % of cells of a pre-determined cell type in the image that comprise one or more labeled biomarkers from within the total number of cells of that cell type observable in the image. In one embodiment the cells of that cell type are determined by the presence of one or more biomarkers. In one embodiment the cells of the that cell type are determined by morphological criteria. In one embodiment the cells of that cell type are determined by a combination of the presence of one or more biomarkers and morphological criteria.

In one embodiment generating the image in b) comprises subtraction of fluorescence emission spectra corresponding to background autofluorescence from the image.

In one embodiment one or more biomarkers are determined in c) to be in high abundance in individuals having a pre-determined disease or condition. In one embodiment one or more biomarkers is determined in c) to be in relatively high abundance in individuals having a pre-determined disease or condition.

In one embodiment a pre-determined disease or condition is a known disease or condition.

In one embodiment a pre-determined disease or condition is a disease or condition for which current detection methods are available but are not clinically applicable.

In one embodiment a pre-determined cell type is a cell type that is known to be associated with a disease or condition.

In one embodiment determining in c) comprises detecting one or more biomarkers on or in cells of the immune system.

In one embodiment determining in c) comprises detecting one or more biomarkers that define a cell type, preferably an immunological cell type.

In one embodiment determining in c) comprises determining that one or more biomarkers are present in high abundance or relatively high abundance in or on cells of the immune system.

In one embodiment the cells of the immune system are selected from the group consisting of T cells, B cells, macrophages, monocytes and dendritic cells.

In one embodiment determining in c) comprises detecting one or more biomarkers on or in at least one tumour cell.

In one embodiment, determining in c) comprises detecting one or more biomarkers at clinically relevant levels.

In one embodiment determining in c) comprises determining that one or more biomarkers are present in high abundance or relatively high abundance in a sample from a subject having a pre-determined disease or condition. In one embodiment the pre-determined disease or condition is an immunological disease or condition. In one embodiment the pre-determined disease or condition is cancer, preferably lung, breast or colorectal cancer and/or melanoma.

In one embodiment the one or more biomarkers are present in relatively high abundance in a sample from an individual suspected of having a pre-determined disease or condition. In one embodiment the pre-determined disease or condition is an immunological disease or condition.

In one embodiment the multispectral image in b) is generated by computer image analysis of fluorescence intensity data from each pixel in the image captured by the multispectral scanner.

In one embodiment the multispectral scanner is the Vectra Polaris.

In one embodiment image analysis of the data generated by the multispectral scanner is performed using In Form cell analysis software or Halo software (PerkinElmer) to generate the image in b).

In one embodiment the image generated in b) comprises at least two unique colours, preferably at least three, at least four, at least five, at least six, at least seven, at least eight, at least nine, at least ten, at least eleven, preferably twelve unique colours, wherein each colour is associated with a different FP fluorescence emission spectra.

In one embodiment the image generated in b) comprises two unique colours, preferably three, four, five, six, seven, eight, nine, ten, eleven, preferably twelve unique colours, wherein each colour is associated with a different FP fluorescence emission spectra.

In one embodiment the image generated in b) comprises at least four, preferably at least five, six, seven or eight unique colours, wherein each colour is associated with a different FP fluorescence emission spectrum.

In one embodiment the image generated in b) comprises four, preferably five, six, seven or eight unique colours, wherein each colour is associated with a different FP fluorescence emission spectrum.

In one embodiment the image generated in b) comprises at least six, seven or eight unique colours, wherein each colour is associated with a different FP fluorescence emission spectrum.

In one embodiment the image generated in b) comprises six, seven or eight unique colours, wherein each colour is associated with a different FP fluorescence emission spectrum.

In one embodiment the image generated in b) comprises six unique colours, wherein each colour is associated with a different FP fluorescence emission spectrum.

In one embodiment the image generated in b) comprises seven unique colours, wherein each colour is associated with a different FP fluorescence emission spectrum.

In one embodiment the image generated in b) comprises eight unique colours, wherein each colour is associated with a different FP fluorescence emission spectrum.

In one embodiment, at least one, two or three unique colours specifically correspond to an FP emission spectrum of about 710 and about 850 nm. In one embodiment at least one unique colour specifically corresponds to a FP maximum emission wavelength of about 753 nm to about 759 nm, preferably of 753 nm, 754 nm, 755 nm or 759 nm.

In one embodiment, one, two or three unique colours specifically correspond to an FP emission spectrum of about 710 and about 850 nm. In one embodiment one unique colour specifically corresponds to a FP maximum emission wavelength of about 753 nm to about 759 nm, preferably of 753 nm, 754 nm, 755 nm or 759 nm.

In another aspect the invention relates to a multispectral immunofluorescence image of a planar biological sample, the image comprising at least three, preferably four, five, six, seven, preferably at least eight colours, wherein at least three, preferably four, five, six, preferably seven colours are associated with the specific binding of at least three, preferably four, five, six, preferably seven Ab-FPs to target antigens comprised in the planar sample.

In one embodiment the multispectral immunofluorescence image of the planar biological sample comprises three, preferably four, five, six, seven, preferably eight colours, wherein three, preferably four, five, six, preferably seven colours are associated with the specific binding of three, preferably four, five, six, preferably seven Ab-FPs to target antigens comprised in the planar sample.

In one embodiment the multispectral image is generated according to a method as described herein.

In one embodiment the multispectral image is generated by computer image analysis of fluorescence intensity data from in each pixel in an image captured by a multispectral scanner according to a method as described herein.

In one embodiment the multispectral scanner is the Vectra Polaris.

In one embodiment the planar biological sample is a tissue section. In one embodiment the tissue section is a frozen or formalin fixed tissue section. In one embodiment the tissue section is a frozen tissue section. In one embodiment the tissue section is a formalin fixed paraffin embedded tissue section.

Specifically contemplated as embodiments of this aspect of the invention relating to a multispectral immunofluorescence image are all of the embodiments set forth in the previous aspects of the invention herein, particularly and not limited to the embodiments relating to antibodies, fluorophores, Ab-FPs, target antigens, biomarkers, tissues, cells, cell types, cellular structures and sub-structures, labeling and multispectral imaging, but not limited thereto.

In another aspect the invention relates to a method of detecting a plurality of target antigens in a biological sample comprising
a) simultaneously labeling at least two target antigens in a planar sample of the biological sample with at least two unique Ab-FP conjugates, wherein the at least two target antigens are present on or in a cell in the sample, and
b) generating a multispectral fluorescence image of the labeled planar sample using a multispectral scanner, wherein the image comprises at least three colours, wherein at least two colours are associated with the specific binding of each unique Ab-FP conjugate to a target antigen, and
c) determining from the image, the presence or absence of the at least two target antigens, each target antigen labeled with a different unique Ab-FP.

In one embodiment the target antigens are different from each other.

In one embodiment the at least two target antigens are present on or in different cell-types in the sample.

In one embodiment, one colour in b) is associated with the fluorescence emission spectra of a nuclear stain. In one embodiment the nuclear stain is DAPI or Hoechst 33342.

In one embodiment labeling in a) comprises simultaneously labeling at least two, preferably at least three, four, five, six, preferably at least seven different target antigens with at least two, preferably at least three, four, five, six, preferably at least seven unique Ab-FP conjugates.

In one embodiment labeling in a) comprises simultaneously labeling two, preferably three, four, five, six, preferably seven different target antigens with two, preferably three, four, five, six, preferably seven unique Ab-FP conjugates.

In one embodiment the multispectral image in b) comprises at least three, preferably at least four, five, six, seven, preferably eight different colours, wherein at least two, preferably three, four, five, six, preferably seven different colours are associated with the specific binding of an Ab-FP to a target antigen.

In one embodiment the multispectral image in b) comprises three, preferably four, five, six, seven, preferably eight different colours, wherein two, preferably three, four, five, six, preferably seven different colours are associated with the specific binding of an Ab-FP to a target antigen.

In one embodiment generating the multispectral image in b) comprises detecting at least two, preferably three, four, five, six, preferably seven different fluorescence spectra respectively using the multispectral scanner, each spectrum detected corresponding to an Ab-FP that is specifically bound to a target antigen.

In one embodiment generating the multispectral image in b) comprises detecting two, preferably three, four, five, six, preferably seven different fluorescence spectra respectively using the multispectral scanner, each spectrum detected corresponding to an Ab-FP that is specifically bound to a target antigen.

In one embodiment labeling in a) comprises simultaneously labeling with at least three Ab-FPs, preferably with at least four, five, six, seven, eight, nine, ten, eleven or preferably at least twelve unique Ab-FPs.

In one embodiment labeling in a) comprises simultaneously labeling with three Ab-FPs, preferably with four, five, six, seven, eight, nine, ten, eleven or preferably at least twelve unique Ab-FPs.

In one embodiment labeling in a) comprises simultaneously labeling with three to seven unique Ab-FPs, preferably six unique Ab-FPs, preferably seven unique Ab-FPs. In one embodiment the planar biological sample is a tissue section. In one embodiment the tissue section is a frozen or formalin fixed tissue section. In one embodiment the tissue section is a frozen tissue section. In one embodiment the tissue section is a formalin fixed paraffin embedded tissue section.

Specifically contemplated herein as embodiments of this aspect of the invention that is a method of detecting a plurality of target antigens are all of the embodiments set forth herein that relate to the previous aspects of the invention set forth herein, including but not limited to antibodies, fluorophores, Ab-FPs, target antigens, biomarkers, tissues, cell types, labeling, multispectral imaging and analysis of multispectral images.

In another aspect the invention relates to a method of detecting a plurality of biomarkers in a biological sample comprising
a) simultaneously labeling at least two target antigens in a planar sample of the biological sample with at least two unique Ab-FP conjugates, wherein each target antigen is comprised by a biomarker in the sample, and
b) generating a multispectral fluorescence image of the labeled planar sample using a multispectral scanner, wherein the image comprises at least three colours, wherein at least two colours are associated with the specific binding of each unique Ab-FP conjugate to a target antigen, and
c) determining from the image, the presence or absence of a plurality of biomarkers, each biomarker comprising a target antigen labeled with a different unique Ab-FP.

In one embodiment determining in c) comprises determining the presence or abundance of a cell type based on the labeling of that cell type with a unique Ab-FP.

Specifically contemplated herein as embodiments of this aspect of the invention that is a method of detecting a plurality of biomarkers are all of the embodiments set forth herein that relate to the previous aspects of the invention set forth herein, including but not limited to antibodies, fluorophores, Ab-FPs, target antigens, biomarkers, tissues, cell types and a method of detecting a plurality of target antigens including labeling, multispectral imaging and analysis of multispectral images, but not limited thereto.

In another aspect the invention relates to a method of detecting a plurality of different cell types in a biological sample comprising
a) simultaneously labeling at least two target antigens in a planar sample with at least two unique Ab-FP conjugates, wherein the target antigens are present on or in a cell in the sample, and
b) generating a multispectral fluorescence image of the labeled planar sample using a multispectral scanner, wherein the image comprises at least three colours, wherein at least two colours are associated with the specific binding of each unique Ab-FP conjugate to a target antigen on a different cell type, and
c) determining from the image, the presence or absence of at least two cell types, each cell type labeled with a different unique Ab-FP.

In one embodiment the cell type is defined by the presence of at least one, preferably two, three, four, five, six or at least seven different biomarkers.

In one embodiment the cell type is defined by the presence of one, preferably two, three, four, five, six or at least seven different biomarkers.

Specifically contemplated herein as embodiments of this aspect of the invention that is a method of detecting a plurality of different cell types in a biological sample are all of the embodiments set forth herein that relate to the previous aspects of the invention, including but not limited to antibodies, fluorophores, Ab-FPs, target antigens, biomarkers, tissues, cell types, and methods of detecting target antigens, biomarkers and cell types including labeling, multispectral imaging and analysis of multispectral images, but not limited thereto.

In another aspect the invention relates to a method of identifying the abundance of a plurality of cell types in a biological sample comprising:
a) simultaneously labeling a planar biological sample with at least two, preferably three, four, five, six, preferably at least seven unique antibody-fluorophore conjugates (Ab-FP), wherein each Ab-FP specifically binds a target antigen on or in a different cell, and
b) generating a multispectral image of the labeled planar sample by simultaneously detecting the fluorescence emission spectra of each FP from each Ab-FP respectively, and
c) determining the abundance of a plurality of different cell types in the sample based on the fluorescence emission spectra detected, optionally with reference to a suitable reference control.

In one embodiment determining in c) comprises subtraction of fluorescence emission spectra corresponding to background autofluorescence from the image generated in b).

In one embodiment abundance is relative abundance.

In one embodiment the relative abundance is the % of cells of a total cell population that are determined to be a cell type.

In one embodiment simultaneously detecting is done using a multispectral scanner, preferably a Vectra Polaris.

Specifically contemplated herein as embodiments of this aspect of the invention that is a method of identifying the abundance of a plurality of different cell types in a biological sample are all of the embodiments set forth herein that relate to the previous aspects of the invention set forth herein, including but not limited to antibodies, fluorophores, Ab-FPs, target antigens, biomarkers, tissues, cell types, and methods of detecting a plurality of target antigens, biomarkers and cell types including labeling, multispectral imaging and analysis of multispectral images, but not limited thereto.

In another aspect the invention relates to a method determining the spatial distribution of a plurality of cell types in a biological sample comprising
a) simultaneously labeling at least two target antigens in a planar sample of the biological sample with at least two unique Ab-FP conjugates, wherein each of the at least two target antigens is present on or in a different cell-type in the sample,
b) generating a multispectral fluorescence image of the labeled planar sample using a multispectral scanner, wherein the image comprises at least three colours, wherein at least two colours are associated with the specific binding of each unique Ab-FP conjugate to a target antigen,
c) identifying from the image at least two different cell types based on the binding of each Ab-FP to the at least two target antigens, and
d) determining from the image, the spatial distribution of a plurality of cell types in a biological sample.

In one embodiment, determining in d) comprises determining the likelihood that a at least one labeled cell type in the image generated in b) is within n cells of one or more different labeled cell types in the image, wherein n=an integer selected from one to ten.

Specifically contemplated herein as embodiments of this aspect of the invention that is a method of determining the spatial distribution of a plurality of cell types in a biological sample are all of the embodiments set forth herein that relate to the previous aspects of the invention set forth herein, including but not limited to antibodies, fluorophores, Ab-FPs, target antigens, biomarkers, tissues, cell types, and methods of detecting a plurality of target antigens, biomarkers and cell types including labeling, multispectral imaging and analysis of multispectral images, but not limited thereto.

In another aspect the invention relates to a method of identifying a patient sub-group from within a group of patients comprising:
a) simultaneously labeling at least three, preferably four, five, six, preferably seven different biomarkers in a planar biological sample from a patient with at least two, preferably three, four, five, six, preferably seven unique antibody-fluorophore conjugates (Ab-FP), wherein each Ab-FP specifically binds a target antigen on a biomarker,
b) generating a multispectral image of the labeled planar sample section by simultaneously detecting the fluorescence emission spectra of each FP in each Ab-FP respectively,
c) detecting the presence or abundance each biomarker in the image generated in b), wherein each biomarker is identified in the image as a different colour that is associated with the specific binding of a unique Ab-FP, and
d) determining from the image that a patient is in a sub-group based on the presence or abundance of each Ab-FP that is specifically bound to each biomarker.

In one embodiment each biomarker is present on a different cell type in the sample.

In one embodiment the patient sub-group is a group of patients having a shared cellular phenotype.

In one embodiment the patient sub-group is defined by the presence of one or more target antigens, biomarkers and/or cell types.

Specifically contemplated herein as embodiments of this aspect of the invention that is a method of identifying a patient sub-group from within a group of patients are all of the embodiments set forth herein that relate to the previous aspects of the invention set forth herein, including but not limited to antibodies, fluorophores, Ab-FPs, target antigens, biomarkers, tissues, cell types, and methods of detecting a plurality of target antigens, biomarkers and cell types including labeling, multispectral imaging and analysis of multispectral images, but not limited thereto.

In another aspect the invention relates to a method of making a diagnostic panel of antibody-fluorophore conjugates (Ab-FPs) comprising:
a) identifying at least three, preferably four, five, six, preferably seven biomarkers for a pre-determined disease or condition,
b) obtaining a unique Ab-FP for each biomarker identified, each Ab-FP comprising an antibody that specifically binds a target antigen on one of the biomarkers identified in a), preferably on each biomarker identified in a), each Ab-FP having a fluorophore (FP) having a maximum fluorescence emission wavelength of about 420 nm to about 850 nm,
c) simultaneously labeling a planar biological sample with the Ab-FPs in b), wherein labeling comprises specifically binding each Ab-FP to a biomarker, preferably wherein each Ab-FP binds a different biomarker, respectively,
d) obtaining a multispectral image of the fluorescence emission spectra of each FP,
e) identifying in the multispectral image the presence or abundance of each biomarker, wherein each biomarker is identified in the image as a different colour that is associated with the specific binding of a different Ab-FP to a target antigen on a biomarker, and
f) selecting the unique Ab-FP conjugates that can be identified in the image in e) as a panel of Ab-FPs that are diagnostic for the pre-determined disease or condition in a).

Specifically contemplated herein as embodiments of this aspect of the invention that is a method of making a diagnostic panel of antibody-fluorophore conjugates are all of the embodiments set forth herein that relate to the previous aspects of the invention set forth herein, including but not limited to antibodies, fluorophores, Ab-FPs, target antigens, biomarkers, tissues, cell types, and methods of detecting a plurality of target antigens, biomarkers and cell types including labeling, multispectral imaging and analysis of multispectral images, but not limited thereto.

In another aspect, the invention relates to a method of identifying a replacement antibody-fluorophore (Ab-FP) conjugate for direct immunofluorescence analysis of a biological sample comprising:
a) generating a first multispectral immunofluorescence image from a single planar biological sample using a first set of at least two to at least seven unique Ab-FP conjugates following a method as described herein, the first multispectral immunofluorescence image comprising up to eight different colours, wherein up to seven colours are each associated with a unique Ab-FP conjugate,
b) selecting at least one of the Ab-FP conjugates from the first set for replacement with a replacement Ab-FP,
c) identifying a suitable antibody for the replacement Ab-FP
d) identifying a suitable fluorophore for the replacement Ab-FP
e) obtaining a replacement Ab-FP,
f) replacing the Ab-FP selected in b) with the replacement Ab-FP to generate a second set of at least two to at least seven unique Ab-FP conjugates,
g) generating a second immunofluorescence image using the second set of Ab-FP conjugates according to a method as described herein, and
h) comparing the first and second multispectral immunofluorescence images,

wherein no difference in the ability to discriminate each different colour in the multispectral image when the first and second images are compared in h) confirms the identification of the replacement Ab-FP conjugate.

Specifically contemplated herein as embodiments of this aspect of the invention that is a method of identifying a replacement antibody-fluorophore (Ab-FP) conjugate for direct immunofluorescence analysis of a biological sample are all of the embodiments set forth herein that relate to the previous aspects of the invention set forth herein, including but not limited to antibodies, fluorophores, Ab-FPs, target antigens, biomarkers, tissues, cell types, and methods of detecting a plurality of target antigens, biomarkers and cell types including labeling, multispectral imaging and analysis of multispectral images, but not limited thereto.

In another aspect, the invention relates to a method for determining if at least one cell type is responsive to a candidate drug; the method comprising:
a) determining the abundance or spatial distribution of at least one cell type in a planar biological sample comprising the at least one cell type using a multispectral immunofluorescence detection method as described herein, and
b) determining if the at least one cell type is responsive to the candidate drug based on the abundance or spatial distribution of the at least one cell type in the sample, optionally in comparison to a suitable control.

In one embodiment the planar biological sample comprises cells cultured in vitro. In one embodiment the planar biological sample comprises a biological tissue or portion thereof.

Specifically contemplated herein as embodiments of this aspect of the invention that is a method for determining if at least one cell type is responsive to a candidate drug are all of the embodiments set forth herein that relate to the previous aspects of the invention set forth herein, including but not limited to antibodies, fluorophores, Ab-FPs, target antigens, biomarkers, tissues, cell types, and methods of detecting a plurality of target antigens, biomarkers and cell types including labeling, multispectral imaging and analysis of multispectral images, but not limited thereto.

In another aspect the invention relates to a method for predicting a treatment response of a patient to a proposed treatment for a pre-determined disease or condition comprising
a) determining the abundance or spatial distribution of at least one cell type in a planar biological sample from the patient using a multispectral immunofluorescence detection method as herein, and
b) determining that the patient will or will not be responsive to the proposed treatment based on the abundance or spatial distribution of the at least one cell type in the sample, optionally in comparison to a suitable control.

Specifically contemplated herein as embodiments of this aspect of the invention that is a method for predicting a treatment response of a patient are all of the embodiments set forth herein that relate to the previous aspects of the invention set forth herein, including but not limited to antibodies, fluorophores, Ab-FPs, target antigens, biomarkers, tissues, cell types, and methods of detecting a plurality of target antigens, biomarkers and cell types including labeling, multispectral imaging and analysis of multispectral images, but not limited thereto.

In another aspect the invention relates to a method of identifying a cellular response to a candidate drug comprising
a) contacting a planar biological sample containing a plurality of cells with the candidate drug,
b) determining the abundance or spatial distribution of at least one cell type in the sample using a multispectral immunofluorescence detection method as herein, and
c) determining from the abundance or spatial distribution of the at least one cell type in sample that there is a cellular response to the candidate drug, optionally by comparison to a suitable control.

Specifically contemplated herein as embodiments of this aspect of the invention that is a method of identifying a cellular response to a candidate drug are all of the embodiments set forth herein that relate to the previous aspects of the invention set forth herein, including but not limited to antibodies, fluorophores, Ab-FPs, target antigens, biomarkers, tissues, cell types, and methods of detecting a plurality of target antigens, biomarkers and cell types including labeling, multispectral imaging and analysis of multispectral images, but not limited thereto.

In another aspect the invention relates to a method of identifying a patient that will benefit from a candidate therapy comprising:
a) labeling a planar biological sample obtained from the subject with at least one unique Ab-FP conjugate, preferably with from one to 12 unique Ab-FP conjugates,
b) obtaining at least one digital fluorescence image of the labeled sample using a multispectral scanner;
c) extracting data associated with at least one emission spectra associated with an Ab-FP conjugate,
d) calculating a distribution function which captures the distribution of data for the at least one emission spectra;
e) deriving a summary score for a patient from the distribution function;
f) evaluating the summary score relative to at least one reference value; selecting the subject as a candidate for a specified therapy based on the summary score, and
g) optionally treating the subject with the specified therapy.

Specifically contemplated herein as embodiments of this aspect of the invention that is a method of identifying a patient that will benefit from a candidate therapy are all of the embodiments set forth herein that relate to the previous aspects of the invention set forth herein, including but not limited to antibodies, fluorophores, Ab-FPs, target antigens, biomarkers, tissues, cell types, and methods of detecting a plurality of target antigens, biomarkers and cell types including labeling, multispectral imaging and analysis of multispectral images, but not limited thereto.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents; or such sources of information, in any jurisdiction, are prior art, or form part of the common general knowledge in the art.

The invention will now be illustrated in a non-limiting way by reference to the following examples.

### EXAMPLES

Materials and Methods

Reagents

Fluorophore-conjugated antibodies listed below were purchased from Biolegend and BD Biosciences (USA):
CD31 BV480
CD141 BB515
CD3 AF532
CD34 PE-CF594
Ki67-AF647
CD21 BB700

Standard materials used in the following examples:
TissueTek OCT compound
Cryomould
Isopentane
Liquid nitrogen
Positive charged slides (for tissue sections)
Ice-cold acetone
DAKO PAP pen
1× TBS
Humidity Chamber
Blocker: 10% human serum prepared in 1× TBS
Antibody dilution buffer: 10% human serum prepared in 1× TBS
DAPI (use at 1:2000 final dilution)
ProlongGold mounting medium
Coverslip

Tissue Samples

Melanoma-infiltrated lymph nodes were provided by our clinical collaborators from

Auckland city hospital.

Sectioning Protocols

(1) Under RNAse free conditions, cut the tissue into blocks of suitable size. If covered in blood or other fluid, blot the tissue on sterile gauze.

(2) Put a small amount of OCT compound into the groove of a cryo-mould. Do not over-fill. Place the tissue centrally on the bottom of the cryo-moulds. If the tissue is too big to fit into a cryo-mould, use an aluminium foil mould.

(3) Fill the mould slowly with Tissue-Tek OCT Compound, avoiding bubble formation, until the tissue is covered by OCT.

(4) Pour about 60 ml of isopentane into a small plastic beaker. This should be enough to cover the tissue block at least twice over.

(5) Lower the isopentane filled plastic beaker into a flask containing liquid nitrogen. The bottom third of the beaker should sit below the surface of the liquid nitrogen. Stir the isopentane occasionally (every 30 sec for 5 sec) with an applicator stick. After about 4 min small white crystals will start forming at the bottom of the beaker. The isopentane is cool enough to use. If liquid nitrogen is not available, the beaker can be placed on dry ice but the isopentane will take longer to cool.

(6) Lower the tissue/OCT filled mould into the isopentane with forceps and hold there until the OCT freezes completely (approx. 7-10 sec). Remove the mould and place in a pre-cooled storage vial.

(7) Store the tissue mould at −80° C. until sectioning.

(8) Section OCT-embedded tissue 5 μm-thick using a cryostat (Leica)

Labeling Protocols

(1) Remove slides with frozen sections from −80° C. freezer and allow slides to warm to RT and dry

(2) Outline slides with DAKO PAP pen

(3) Fix tissue sections with ice-cold acetone for 5 min at RT. Acetone should evaporate and the slide dry.

(4) Briefly wash slides with TBS

(5) Incubate tissue sections with a blocker at RT for 10 min in a humidity chamber in a dark place

(6) Flick off blocker

(7) Add fluorophore-conjugated antibodies prepared in antibody dilution buffer to tissue sections simultaneously and incubate for 1 hour in a humidity chamber in a dark place at RT (protected from light)

(8) Wash briefly ×1 with TBS, followed by 3× 5 min washes with TBS on a rocker

(9) Add DAPI (1:2000) to tissue sections and incubate for 5 min at RT in a humidity chamber in a dark place (protected from light)

(10) After washing 3× 2 min washes with TBS on a rocker, mount slides with a coverslip using ProlongGold.

(11) Proceed with imaging using Vectra Polaris.

Scanning Protocols (from Vectra Polaris User Manual 1.0.7)

(1) Turn on the Vectra Polaris Instrument and computer

(2) Launch the Vectra Polaris software

(3) Load slides into the slide carriers

(4) Load slide carriers into the slide carrier hotel for microscope slide scanning

(5) In the ‘Edit protocol’ page (Vectra Polaris software), create a protocol for imaging. Select the fluorescent mode and spatial resolution (typically ×20 magnification, also available at ×10 or ×20) for the whole slide scanning (WSS) and for multispectral imaging (MSI) of regions of interest (ROIs). Also set the exposure times for WSS and MSI and what filters to use for focusing and imaging.

(6) In the ‘Scan slide’ page (Vectra Polaris software), locate the slides to be scanned and perform the whole slide scan (WSS) using the WSS protocol created in (5)

(7) Launch the Phenochart program (PerkinElmer) to view the WSS image and select ROIs for multispectral imaging (MSI).

(8) In the ‘Scan slide’ page (Vectra Polaris software), locate the slides containing selected ROIs to be imaged in a multispectral manner. Perform multispectral imaging (MSI) of selected ROIs using the MSI protocol created in (5)

(9) After imaging selected ROIs, unmix acquired MSI images using spectral libraries built from images of single stained tissues for each Ab-FP in the InForm software (PerkinElmer). Process and analyze unmixed images in the InForm software

### Example 1

The following protocol was used to specifically detect six different target antigens in a frozen section of a tumour tissue using Ab-FPs as described herein.

Following the methods of the invention as described herein a seven-colour multiplex immunofluorescence image was generated (6 Abs+DAPI).

Thus, following the methods as described herein allows rapid and specific multiplex detection of multiple target antigens without the need for a secondary antibody incubation step.

Six antibodies are added simultaneously in this protocol, while one primary antibody is added at a time in seven colour opal protocol (FFPE). Accordingly, this example of a method of the invention illustrates that the distinct advantage provided that is a greatly reduced overall time required for specific detection and identification of multiple target antigens.

This example of the method of the invention employs frozen tissue, so there is no need for dewaxing and antigen retrieval.

Materials
Frozen tissue slides
Ice-cold acetone
DAKO PAP pen
1× TBS
Humidity Chamber
Blocker: 10% human serum prepared in 1× TBS
Dilution buffer: 10% human serum prepared in 1× TBS
Fluorophore-conjugated antibodies
   CD31 BV480
   CD141 BB515
   CD3 AF532
   CD34 PE-CF594
   Ki67 AF647
   CD21 BB700
DAPI (use at 1:2000 final dilution)
ProlongGold mounting medium
Coverslip

Methods
1. Select slides:
   Remove slides with frozen sections from −80° C. freezer.
   Allow slides to warm to RT and dry
2. Outline slide with DAKO PAP pen
3. Fix with ice cold acetone at RT for 5 min. Acetone should evaporate and the slide dry.
4. Wash briefly with 1× TBS
5. Block with 10% human AB serum blocker at RT for 10 min in a humidity chamber
6. Prepare Ab cocktails in dilution buffer (with 10% human serum).
7. Flick off blocker over sink.
8. Incubate section with Ab cocktails simultaneously at RT for 1 hr in a humidity chamber in a dark place
9. Flick off Ab cocktails
10. Wash briefly ×1 with TBS followed by 3× 5 min washes with TBS on a rocker
11. Add DAPI (final concentration at 1:2000) and incubate for 5 min at RT in a humidity chamber in a dark place
12. After washing 3× 2 min washes with TBS on a rocker, mount the slides with ProlongGold
13. Image slides using Vectra Polaris

Results

Frozen tissue section from melanoma-infiltrated lymph node was fixed with acetone, blocked with 0.25% casein+10% human serum, and labeled with six antibodies conjugated to different fluorophores simultaneously including anti-CD31 BV480, anti-CD141 BB515, anti-CD3 AF532, anti-CD34 PE-CF594, anti- Ki67-AF647, and anti-CD21 BB700 for 1 hr at RT. After washing, tissue section was then labeled with DAPI for 5 min at RT and mounted. Subsequently, tissue section was scanned over the whole slide using the multispectral scanner of the Vectra Polaris and regions of interest (ROIs) were selected and imaged multispectrally. Acquired images were unmixed in the InForm software (PerkinElmer) (FIGS. 1 to 6).

The ability of the methods disclosed herein to simultaneously detect the presence and abundance of a plurality of target antigens, biomarkers and cell types is elegantly illustrated in FIGS. 1 to 13 which show the distribution of different immune and stromal cell populations within a melanoma-infiltrated lymph node tissue section simultaneously labeled with DAPI and six Ab-FPs as described herein.

### Example 2

The following protocol was substantially the same as set out for Example 1 and was used to specifically detect seven different target antigens in a frozen section of a tumour tissue using Ab-FPs as described herein.

Following the methods of the invention as described herein an eight-colour multiplex immunofluorescence image was generated (7 Abs+DAPI).

As in Example 1, all Ab-FPs (seven) were added simultaneously in this protocol illustrating again the distinct advantage disclosed herein of greatly reduced overall time required for specific detection and identification of multiple target antigens in a single sample.

This example of the method of the invention also employs frozen tissue, so there is no need for dewaxing and antigen retrieval.

Materials
Frozen tissue slides
Ice-cold acetone
DAKO PAP pen
1× TBS
Humidity Chamber
Blocker: 10% human serum prepared in 1× TBS
Dilution buffer: 10% human serum prepared in 1× TBS
Fluorophore-conjugated antibodies
   CD31 BV480
   CD141 BB515
   CD3 AF532
   CD34 PE-CF594
   Ki67 AF647
   CD21 BB700
   CD11c AF700
DAPI (use at 1:2000 final dilution)
ProlongGold mounting medium
Coverslip

Methods
1. Select slides:
   Remove slides with frozen sections from −80° C. freezer.
   Allow slides to warm to RT and dry
2. Outline slide with DAKO PAP pen
3. Fix with ice cold acetone at RT for 5 min. Acetone should evaporate and the slide dry.
4. Wash briefly with 1× TBS
5. Block with 10% human AB serum blocker at RT for 10 min in a humidity chamber
6. Prepare Ab cocktails in dilution buffer (with 10% human serum).
7. Flick off blocker over sink.
8. Incubate section with Ab cocktails simultaneously at RT for 1 hr in a humidity chamber in a dark place
9. Flick off Ab cocktails
10. Wash briefly ×1 with TBS followed by 3× 5 min washes with TBS on a rocker
11. Add DAPI (final concentration at 1:2000) and incubate for 5 min at RT in a humidity chamber in a dark place
12. After washing 3× 2 min washes with TBS on a rocker, mount the slides with ProlongGold
13. Image slides using Vectra Polaris

Results

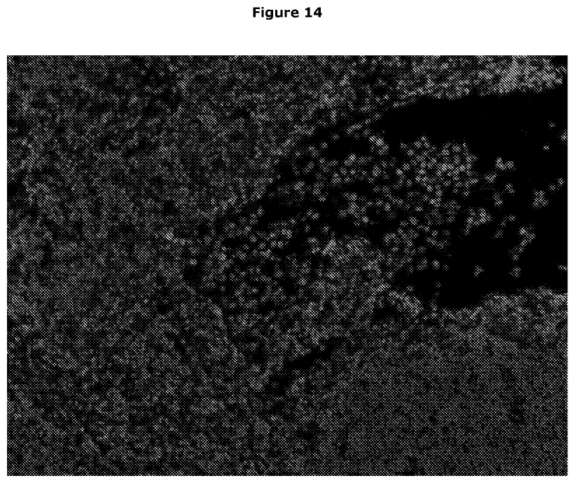

Frozen tissue section from melanoma-infiltrated lymph node was fixed with acetone, blocked with 0.25% casein+10% human serum, and labeled with seven antibodies conjugated to different fluorophores simultaneously including anti-CD31 BV480, anti-CD141 BB515, anti-CD3 AF532, anti-CD34 PE-CF594, anti-Ki67-AF647, anti-CD11c AF700 and anti-CD21 BB700 for 1 hr at RT. After washing, tissue section was then labeled with DAPI for 5 min at RT and mounted. Subsequently, tissue section was scanned over the whole slide using the multispectral scanner of the Vectra Polaris and regions of interest (ROIs) were selected and imaged multispectrally. Acquired images were unmixed in the InForm software (PerkinElmer) (FIGS. 14-23).

The ability of the methods disclosed herein to simultaneously detect the presence and abundance of a plurality of target antigens, biomarkers and cell types in frozen tissue sections is elegantly illustrated in FIGS. 1 to 25 which show the distribution of different immune and stromal cell populations within a melanoma-infiltrated lymph node tissue section simultaneously labeled with DAPI and six or seven Ab-FPs as described herein.

### Example 3

The following protocol was substantially the same as set out for Example 1 and was used to specifically detect CD163+ cells in a frozen section of a tumour tissue using a CD163 APC/Fire750 Ab-FP conjugate as described herein.

Following the methods of the invention as described herein an unmixed immunofluorescence image was generated to further illustrate the ability of the methods disclosed herein to detect Ab-FP conjugates comprising an FP that emits in the far-red end of the spectrum.

As in Example 1, the Ab-FP conjugate was used to directly label tissue, added further illustrating a distinct advantage of the present disclosure of specific detection of antigen expression by direct detection (as compared to indirect or secondary detection) using a single Ab-FP conjugate to identify a target antigen in a tissue sample.

This example of the method of the invention also employs frozen tissue, so there is no need for dewaxing and antigen retrieval.

Materials
Frozen tissue slides
Ice-cold acetone
DAKO PAP pen
1× TBS
Humidity Chamber
Blocker: 10% human serum prepared in 1× TBS
Dilution buffer: 10% human serum prepared in 1× TBS
Fluorophore-conjugated antibodies
   CD163 APC/Fire750
ProlongGold mounting medium
Coverslip

Methods
1. Select slides:
   Remove slides with frozen sections from −80° C. freezer.
   Allow slides to warm to RT and dry
2. Outline slide with DAKO PAP pen
3. Fix with ice cold acetone at RT for 5 min. Acetone should evaporate and the slide dry.
4. Wash briefly with 1× TBS
5. Block with 10% human AB serum blocker at RT for 10 min in a humidity chamber
6. Prepare Ab in dilution buffer (with 10% human serum).
7. Flick off blocker over sink.
8. Incubate section with Ab at RT for 1 hr in a humidity chamber in a dark place
9. Flick off Ab
10. Wash briefly ×1 with TBS followed by 3× 5 min washes with TBS on a rocker
11. Mount the slides with ProlongGold
12. Image slides using Vectra Polaris

Results

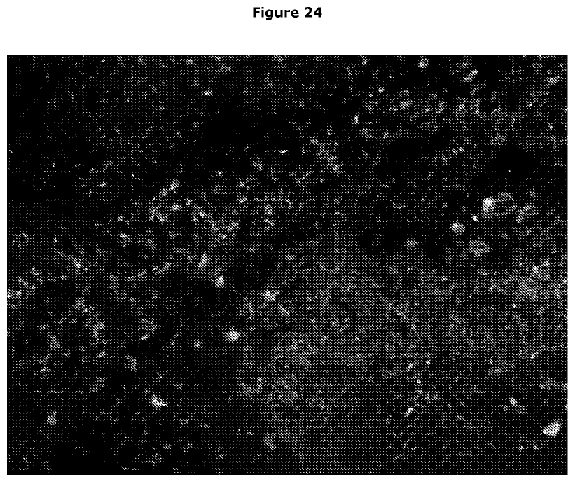

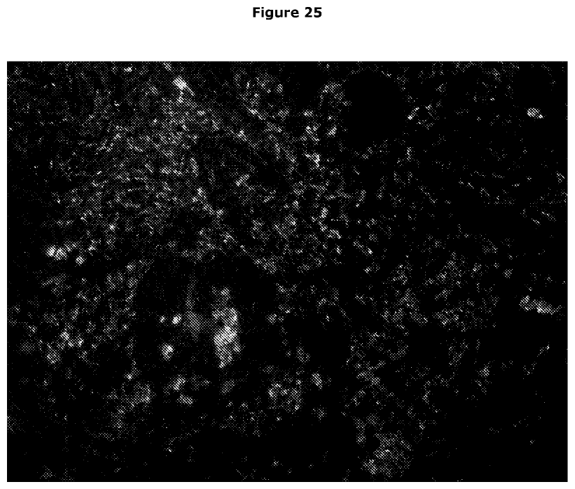

Frozen tissue section from melanoma-infiltrated lymph node was fixed with acetone, blocked with 0.25% casein+10% human serum, and labeled with a single Ab-FP conjugate; anti-CD163 APC/Fire750 for 1 hr at RT. After washing, the tissue section was mounted. Subsequently, tissue section was imaged multispectrally using the multispectral scanner Vectra Polaris Acquired images were unmixed in the InForm software (PerkinElmer) (FIGS. 24 and 25).

The ability of the methods disclosed herein to detect the presence and abundance of a target antigens, biomarkers and cell types by direct labeling using an Ab-FP conjugates comprising fluorophores that emit in the far-red end of the spectrum as described herein is elegantly illustrated in FIGS. 24 and 25. These figures show the distribution of CD163+ cell populations within a melanoma-infiltrated lymph node tissue section.

### Example 4

The following protocol was used to specifically detect two different target antigens in sections of formalin fixed paraffin embedded (FFPE) tonsil tissue using Ab-FPs as described herein. The following Ab-FPs were used: CD45RO-AF488, CD19-AF647 and CD8-AF647.

Following the methods of the invention as described herein a three-colour multiplex immunofluorescence image was generated (2 Abs+DAPI).

Thus, following the methods as described herein allows rapid and specific multiplex detection of multiple target antigens in FFPE tissue sections without the need for a secondary antibody incubation step.

In comparison to known methods of multiplex immunofluorescence detection, the methods described herein allow for a more rapid examination of marker co-localization within in the same subcellular compartments. The described methods also generate higher resolution images due to the reduced diffusion of fluorescence emission from the fluorophore in the Ab-FP, which is due to the direct conjugation of the fluorophore to the primary antibody.

In the described methods, two primary antibodies are added simultaneously in a single step staining protocol. In contrast, only a single primary antibody is added in multiple different steps to obtain a multi-colour image following an Opal protocol (FFPE). Accordingly, the method described herein provides at least one distinct advantage over known methods in greatly reducing the overall time required for specific immunofluorescence detection and identification of multiple target antigens in a single tissue section, particularly in FFPE tissue sections.

Materials and Methods

Reagents

Fluorophore-conjugated antibodies listed below were purchased Biolegend and BD Biosciences:
CD19 AF647
CD8 AF647
CD45RO AF488
CD45RO AF594
CD45RO AF700
CD45RO BV510
CD45RO BV650
CD45RO PE/Dazzle594
CD45RO APC/Fire750

Standard materials used in the following examples:
Positive charged slides (for tissue sections)
Coverslip
Baths and solvents for deparaffinization and rehydration of FFPE tissue
Retriever 2100 (for antigen retrieval)
Xylene
Ethanol
H₂O
10% NBF
DAKO PAP pen
1× Antigen-retrieval buffers
1× TBS
Humidity Chamber
Blocker: 0.25% casein+10% human serum prepared in 1× TBS
Antibody dilution buffer: 10% human serum prepared in 1× TBS
DAPI (use at 1:2000 final dilution)
ProlongGold mounting medium

Tissue Samples

Formalin-fixed paraffin-embedded tonsil tissue was provided by our clinical collaborators from Auckland city hospital.

Tissue Preparation and Staining Protocols

(1) Bake slides in an oven, temperature set at 60° C., for at least 1 hour up to over-night

(2) De-waxing and rehydration: load slides into staining rack and process with the treatments as listed below:
Xylene: 3× 10 min
99% Ethanol: 2× 5 min
90% EtOH: 1× 10 min
70% EtOH: 30 sec
dH2O: 30 sec
10% NBF: 1× 10 min
dH2O: 30 sec

(3) Load slides into slide chambers containing 1× Antigen-retrieval buffers

(4) Place the slide chambers in the Retriever 2100 filled with dH2O. Press start

(5) After cooling the slides, rinse with dH2O five times and wash in 1× TBS for 5 min twice

(6) Wipe away liquid around sections with paper tissue. Circle sections with PAP pen to restrict the area

(7) Incubate tissue sections with 10% HS blocker at RT for 10 min in a humidity chamber

(8) Flick off blocker

(9) Add fluorophore-conjugated antibodies prepared in antibody dilution buffer to tissue sections simultaneously and incubate for 1 hour in a humidity chamber in a dark place at RT (protected from light)

(10) Flick off Abs over sink. Wash briefly ×1 with TBS, followed by 3× 5 min washes with TBS on a rocker

(11) Add DAPI (1:2000) to tissue sections and incubate for 5 min at RT in a humidity chamber in a dark place (protected from light)

(12) After washing 3× 2 min washes with TBS on a rocker, mount slides with a coverslip using ProlongGold.

(13) Proceed with imaging using Vectra Polaris.

Scanning Protocols (from Vectra Polaris User Manual 1.0.7)

(1) Turn on the Vectra Polaris Instrument and computer

(2) Launch the Vectra Polaris software

(3) Load slides into the slide carriers

(4) Load slide carriers into the slide carrier hotel for microscope slide scanning

(5) In the ‘Edit protocol’ page (Vectra Polaris software), create a protocol for imaging. Select the fluorescent mode and spatial resolution (typically ×20 magnification, also available at ×10 or ×40) for the whole slide scanning (WSS) and for multispectral imaging (MSI) of regions of interest (ROIs). Also set the exposure times for WSS and MSI and what filters to use for focusing and imaging.

(6) In the ‘Scan slide’ page (Vectra Polaris software), locate the slides to be scanned and perform the whole slide scan (WSS) using the WSS protocol created in (5)

(7) Launch the Phenochart program (PerkinElmer) to view the WSS image and select ROIs for multispectral imaging (MSI).

(8) In the ‘Scan slide’ page (Vectra Polaris software), locate the slides containing selected ROIs to be imaged in a multispectral manner. Perform multispectral imaging (MSI) of selected ROIs using the MSI protocol created in (5)

(9) After imaging selected ROIs, unmix acquired MSI images using spectral libraries built from images of single stained tissues for each Ab-FP in the InForm software (PerkinElmer). Process and analyze unmixed images in the InForm software

Results

Formalin fixed paraffin embedded (FFPE) tissue sections from tonsils were deparaffinized in xylene, rehydrated, and heat-treated in the retriever with antigen-retrieval buffer. Tissue sections were then blocked with 0.25% casein+10% human serum, and labeled with Ab-FP conjugates and DAPI. Subsequently, tissue sections were imaged multispectrally using the multispectral scanner Vectra Polaris Acquired images were unmixed in the InForm software (PerkinElmer).

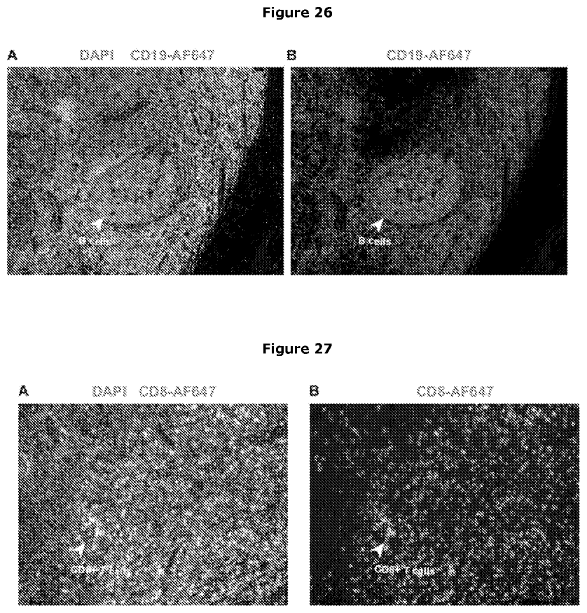

The ability of the methods disclosed herein to simultaneously detect the presence and abundance of a plurality of target antigens, biomarkers and cell types in FFPE tissue sections is elegantly illustrated in FIGS. 26 to 36 which show the distribution of antigen and cell populations within tonsil tissue sections simultaneously labeled with DAPI and one or two Ab-FPs as described herein.

### INDUSTRIAL APPLICATION

The antibody-fluorophore conjugates and methods of using such of the invention have industrial application in molecular biology in providing a means to diagnose and manage disease including cancer.

### REFERENCES

Gorris, M., et al. (2018). Eight-Color Multiplex Immunohistochemistry for Simultaneous Detection of Multiple Immune Checkpoint Molecules within the Tumor Microenvironment. J Immunol., 200(1), 347-354. doi:10.4049/jimmunol.1701262
Hofman, P., et al. (2019). Multiplexed Immunohistochemistry for Molecular and Immune Profiling in Lung Cancer—Just About Ready for Prime-Time? Cancers (Basel), 11(3):283. doi:10.3390/cancers11030283
Majtahed, A., et al. (2011). A two-antibody mismatch repair protein immunohistochemistry screening approach for colorectal carcinomas, skin sebaceous tumors, and gynecologic tract carcinomas. Modern Pathology, 24, 1004-1014.
Sood, A., et al. (2016). Multiplexed immunofluorescence delineates proteomic cancer cell states associated with metabolism. JCI Insight, 1(6). doi:10.1172/jci.insight.87030

1. A composition comprising at least five, six or seven antibody-fluorophore conjugates (Ab-FP), wherein each FP has a different fluorescence excitation and emission spectra (Ex).

2-4. (canceled)

5. The composition of claim 1, wherein each FP has a maximum excitation and emission wavelength (Ex/Em) selected from the group consisting of 348/395, 404/448, 405/421, 405/510, 405/570, 405/603, 405/646, 405/711, 407/421, 415/500, 436/478 nm, 490/515 nm, 494/520 nm, 495/519 nm, 485/693 nm, 496/578, 532/554 nm, 566/610 nm, 590/620 nm, 650/660 nm, 650/668 nm, 652/704, 696/719 nm, 753/785 nm, 754/787 nm, 755/775 nm and 759/775 nm.

6-7. (canceled)

8. The composition of claim 1, wherein the FP is selected from the group consisting of Brilliant™ Ultraviolet 395 (BUV395) having an Ex/Em of 348/395, Brilliant™ Violet 480 (BV480) having an Ex/Em of 436/478 nm, Brilliant Violet 421™ having an Ex/Em of 405/421, Brilliant™ Violet 421 (BV421) having an Ex/Em of 407/421, Brilliant™ Violet 510 (BV510) having an Ex/Em of 405/510, Brilliant Violet 570™ having an Ex/Em of 405/570, Brilliant Violet 605™ having an Ex/Em of 405/603, Brilliant Violet 650™ having an Ex/Em of 405/646, Brilliant Violet 711™ having an Ex/Em of 405/711, BD Horizon™ V450 having an Ex/Em of 404/448, BD Horizon™ V500 having an Ex/Em of 415/500, Brilliant™ Blue 515 (BB515) having an Ex/Em of 490/515 nm, Fluorescein Isothiocyanate (FITC) having an Ex/Em of 494/520 nm, Alexa Fluor 488 (AF488) having an Ex/Em of 495/519 nm, Alexa Fluor 532 (AF532) having an Ex/Em of 532/554 nm, R-phycoerythrin (PE) having an Ex/Em of 496/578, Alexa Fluor 594 (AF594) having an Ex/Em of 590/620 nm, PE-Dazzle 594 (PE594) or PE-CF594 (CF594) having an Ex/Em of 566/610 nm, Alexa Fluor 647 (AF647) having an Ex/Em of 650/668 nm, Allophycocyanin (APC) having an Ex/Em of 650/660, BD Horizon™ 700 (BB700) having an Ex/Em of 485/693 nm, Alexa Fluor 700 (AF700) having an Ex/Em of 696/719 nm, APC/Alexa Fluor 750 having an Ex/Em of 753/785 nm, APC/Fire 750 having an Ex/Em of 754/787 nm, APC-R700 having an Ex/Em of 652/704, APC-Cy7 having an Ex/Em of 755/775 nm and AF750 having an Ex/Em of 759/775 nm.

9. (canceled)

10. The composition of claim 1, wherein the Abs in the Ab-FP are selected from the group consisting of anti-CD31, CD141, CD144, CD3, CD34, CD163, CD11c, CD14, CD16, CD68 Foxp3, CD4, CD8, CD19, CD20, CD25, CD38, PD-1, PDL1, PDL2, CD68, Ki-67, Sox10, S100, PRAME, MART1 and anti-CD21 antibodies.

11. (canceled)

12. The composition of claim 1, comprising at least five, six or all seven of the following antibodies: anti-CD31, CD141, CD3, CD34, Ki-67, CD11c and anti-CD21 antibodies.

13. The composition of claim 1, comprising at least five, six or preferably all seven of the following Ab-FPs: CD31-BV480; CD141-BB515; CD3-AF532; CD34-PE-CF594; Ki67-AF647, CD11c-AF700 and CD21-BB700.

14. The composition of claim 1, wherein the Ab in the Ab-FP comprises one or more Ab selected from the group consisting of anti-oestrogen receptor (ER), progesterone receptors (PR), her2 and anti-cytokeratin antibodies.

15. (canceled)

16. The composition of claim 1, wherein the Ab in the Ab-FP comprises one or more antibodies selected from the group consisting of anti-b-raf (V600E mutation), MLH1, MSH2, MSH6, and anti-PMS2 antibodies, wherein each Ab specifically binds a target antigen.

17-19. (canceled)

20. The composition of claim 16, wherein the target antigen is a T cell, B cell, macrophage, monocyte or dendritic cell antigen.

21-24. (canceled)

25. A method of direct immunofluorescence analysis of biological sample comprising
a. labeling at least one target antigen in a planar biological sample with at least one unique Ab-FP conjugate, and
b. generating a multispectral fluorescence image of the labeled planar sample using a multispectral scanner, wherein the image comprises at least two colours, wherein at least one colour is associated with the specific binding of the at least one unique Ab-FP conjugate to the at least one target antigen, and
c. determining from the image the presence or absence of at least one biomarker comprising the at least one target antigen.

26-29. (canceled)

30. The method of claim 25, wherein generating the multispectral image in b) comprises detecting at least five, six, or seven different fluorescence spectra respectively using the multispectral scanner, wherein each spectrum detected corresponds to an Ab-FP that is specifically bound to a target antigen.

31-35. (canceled)

36. The method of claim 25, wherein labeling in a) comprises labeling with at least five, six or all seven of the following antibodies: anti-CD31, CD141, CD3, CD34, Ki67, CD11c and anti-CD21 antibodies.

37. The method of claim 25, wherein labeling in a) comprises labeling with at least five, six, or all seven of the following Ab-FPs: CD31-BV480; CD141-BB515; CD3-AF532; CD34-PE-CF594; Ki67-AF647; CD11c-AF700 and CD21-BB700.

38-41. (canceled)

42. The method of claim 25, wherein determining in c) is detecting the presence of at least five, six, or seven different biomarkers.

43. (canceled)

44. The method of claim 25, wherein determining in c) comprises detecting one or more biomarkers on or in cells of the immune system wherein the cells of the immune system are selected from the group consisting of T cells, B cells, macrophages, monocytes and dendritic cells.

45-47. (canceled)

48. The method of claim 25 wherein the image generated in b) comprises at least five, at least six, at least seven, at least eight, at least nine, at least ten, at least eleven, preferably twelve unique colours, wherein each colour is associated with a different FP fluorescence emission spectra.

49. (canceled)

50. The method of claim 25 wherein the image generated in b) comprises at least six or seven unique colours one, two or three unique colours that specifically correspond to an FP emission spectrum of about 710 and about 850 nm, or to a FP maximum emission wavelength of about 753 nm to about 759 nm, or of 753 nm, 754 nm, 755 nm or of 759 nm.

51. (canceled)
